(19) United States
(12) Patent Application Publication
(10) Pub. No.: US 2025/0073429 A1
(43) Pub. Date: Mar. 6, 2025

(54) CATHETER CONSTRUCTION

(71) Applicant: Maduro Discovery, LLC, Campbell, CA (US)

(72) Inventor: Brian B. MARTIN, Campbell, CA (US)

(73) Assignee: Maduro Discovery, LLC, Campbell, CA (US)

(21) Appl. No.: 18/817,892

(22) Filed: Aug. 28, 2024

Related U.S. Application Data
(60) Provisional application No. 63/579,163, filed on Aug. 28, 2023.

(51) Int. Cl. A61M 25/10; A61M 25/00

(52) U.S. Cl. CPC A61M 25/10; A61M 25/0045; A61M 2025/006; A61M 2025/1088; A61M 2205/0216

(57) ABSTRACT

Polymeric tubing, for use with catheters or other medical devices, where the polymeric tubing includes one or more functional lumens within a wall of the tubing.

### CROSS-REFERENCE TO RELATED APPLICATIONS

This application is a non-provisional of U.S. Provisional Application No. 63/579,163 filed Aug. 28, 2023. The entirety of which is incorporated by reference.

### FIELD OF THE INVENTION

Polymeric tubing for use with catheters or other medical devices, where the polymeric tubing can have lengths of customized properties including, but not limited to, durometer, torque control, flexibility, axial strength, stiffness, etc. In one variation, the transition regions between lengths can be configured such that there can be abrupt, gradual, or customized transition regions between various lengths such that the structural characteristics differential between the lengths and over the transition regions are selectively designed. In certain variations, the structural characteristic differential is minimized or eliminated as compared to conventional catheters. The devices described herein can also include functional lumens within the materials sections that form all or a portion of a wall of the tube, where such materials extend in a spiral direction about the polymeric tubing.

### BACKGROUND OF THE INVENTION

Balloon catheters are commonly used in medical procedures and often for minimally invasive intravascular procedures. These balloon catheters can be used to occlude a vessel or other region of the anatomy, dilate stenosed vessels, provide drug therapy, as well as perform a number of other medical treatments.

FIG. 1A illustrates a common design for a conventional balloon catheter 2 having an inner tube/or extrusion 4 within an outer tube/extrusion 5 with a balloon membrane 6 joined to both tubes 4 and 5. As shown in FIG. 1A, the balloon membrane 6 (referred to as a balloon) comprises a soft material that can expand, either elastically or inelastically, when pressurized. FIG. 1B illustrates a cross-sectional view of the catheter in FIG. 1A, showing a gap 7 between the inner 4 and outer tube 5 that allows for increasing pressure to cause expansion of the balloon 6. FIG. 1C illustrates a cross-sectional view of another conventional design where tube 5 comprises a dual lumen configuration having an inner or working lumen 9.

Regardless of the design, conventional balloon catheters are difficult to navigate into remote distal vasculature because of the tortuous path of the vasculature. Conventional balloon catheter construction is not optimized for improved navigation through difficult anatomy.

FIG. 1D illustrates a general example of traditional catheter construction that can be used with conventional balloon catheters. FIG. 1D shows a sectional view of a traditional catheter section 10 constructed over an inner mandrel or core 12 that is later removed. The traditional catheter construction includes a layer 14, such as PTFE, that provides a lubricious surface for the interior of the catheter while also supporting various structural components to provide varying sections 16 and 18 of the catheter 10. For example, the illustrated catheter 10 includes a reinforced section 16 in which a braid or coil 20 (or both) is wrapped around the second layer 14. Many catheters use metal braids in the proximal end of the catheter and metal coils in the distal end of the catheters (or one under the other).

Conventional catheters that are intended to navigate through tortuous anatomy also include regions with varying durometers 18 in which polymers of different durometers 22, 24, and 26 are placed next to each other. FIG. 1A is intended for illustrative purposes to show the basic structures of conventional catheters. The catheter 10 of FIG. 1A shows polymer 22 terminates before a distal end 8 of the catheter 10 for illustrative purposes to only show the underlying reinforced section 16. In most conventional catheters, the entirety of the distal end is encapsulated by a polymer.

As shown in FIG. 1D, a series of adjacently placed polymer jackets 22, 24, 26 are placed over the reinforcement layer and fused into place (such as by heating and reflowing the polymer onto the braid or coil). Different polymer durometers (i.e., “stiffness”) are used for different sections. As a result, each of these sections of catheter will have unique structural characteristics/properties, where the structural properties can include but are not limited to stiffnesses, resistance to twisting or torsion, flexibility, column strength, etc. The illustrated construction 10 provides for varying structural characteristics over the varying regions of the catheter. However, in conventional devices, such a catheter construction yields abrupt changes in characteristics at the transition or edge of each region 22, 24, 26.

In conventional catheter design, higher durometer polymers are used in the proximal region, with softer durometers placed as the catheter progresses toward the distal end. More sophisticated catheters have more “sections” or transitions of stiffness. Typically, stiffer durometers are more suitable for the proximal region of the catheter. Although stiffer durometer polymers do not bend as well around curves, they have greater positional stability in the vessels and tend to transmit torque well. In contrast, softer durometers are suitable for the distal region of the catheter because these polymers bend more easily and gently around the more delicate and tortuous distal curves. However, softer durometer polymers do not transmit torque well and have poor positional stability. Thus, conventional catheter designs use a “balancing act” between mechanical properties, where the design elements (stiff and stable vs. soft and less stable) are compromised. Additionally, the change from one durometer to another has long been a source of mechanical challenge. These transitions are a source of discontinuity and are known in the field to cause challenges in torque transmission and can lead to irregularities in bending stress, which leads to poor navigation in the anatomy. As such, engineers attempt to make the transitions as long and gradual as possible and to mitigate abrupt changes by having numerous small transitions as opposed to fewer larger transitions.

Regardless of the length of the transitions, the traditional construction, as shown in FIG. 1D relies on the braid or coil 20 (or both) used to transmit torque as the catheter navigates through tortuous anatomy. However, since the polymers 22, 24, 26 (etc.) are exterior to the braid/coil 20, a greater degree of torque is applied to the polymers. Polymers having different physical properties will also have different resistance to torque. For example, in a variation where polymers 22, 24, and 26 have decreasing flexibility (22 being the most flexible and 26 being the least), torque applied by the rotation of section 26 will not be fully applied to section 24. Therefore, section 24 will not rotate as much as section 26. The same effect will occur with section 22; its rotation will not be as much as section 24 and even less than section 26. This results in poor torque control or torque instability. Furthermore, when these sections are flexed, the transition between polymers creates discontinuities in how the catheter responds to flexing or bending across different sections.

In view of the above, for conventional catheters, there is a limit to the number of constructional variations or “design levers” that can be changed to accommodate changing properties of the catheter in different regions. Such conventional design levers include varying the durometer of the polymer, reinforcement braid, and coil design, transition length, etc. However, Radical Catheter Technologies has recently developed a catheter construction that increases the number of available design levers by an order of magnitude over number of design levers for conventional catheter construction. These improved catheter constructions are discussed in U.S. Pat. No. 11,077,285 issued Aug. 3, 2021, WO2020257125, U.S. Pat. No. 10,898,683 issued Jan. 26, 2021, U.S. Pat. No. 11,179,546 issued Nov. 23, 2021, US20210290907, PCT application PCT/US2023/071362, and U.S. Provisional application 63/514,208, the entirety of each of which is incorporated by reference.

Accordingly, there remains a need to improve balloon catheter design using the recently developed improved catheter construction.

### SUMMARY OF THE INVENTION

Variations of the present disclosure include a catheter tubing including: a tubular structure extending along an axial length and having a passageway; a tubular layer forming at least a portion of a wall of the tubular structure and including a plurality of material sections extending spirally along at least a portion of the axial length, where each material section is joined to an adjacent material section; wherein the plurality of material sections include at least a first material section and a second material section, the first material section includes a first structural property and the second material section includes a second structural property, where the first structural property differs from the second structural property; at least one lumen extending within at least one of the plurality of material sections, where the at least one lumen permits fluid flow therethrough.

The catheter tubing can further include at least one opening in the tubular layer, where the at least one opening is fluidly coupled to the at least one lumen.

In additional variations, the catheter tubing can have a balloon comprised of a balloon material over the at least one opening, where the balloon material is affixed to the tubular layer such that pressurization of the at least one lumen causes expansion of the balloon.

In an additional variation, a medical tubing can include an elongate body extending along an axial length; and a plurality of material sections extending spirally along at least a first portion of the axial length to form at least a portion of a wall of the elongate body, the plurality of material sections including a first material section; wherein the first material section includes a first functional lumen extending therein, such that pressurization of the first functional lumen causes the first material section to expand radially outward from the plurality of material sections to form an expanded spiral shape on the elongate body.

In another variation, a catheter tubing can have a tubular structure extending along an axial length and having a passageway; a composite layer forming at least a portion of a wall of the tubular structure and including a plurality of tubing extensions extending spirally along at least a portion of the axial length, where at least one of the plurality of tubing extensions includes a lumen extending therein.

Another variation of a tubing can include a tubular structure having a wall and a passageway extending in the tubular structure; and a material section extending spirally in a portion of the wall over a first axial length, where the material section includes a functional lumen extending therein along the first axial length, where the functional lumen is fluidly isolated from the passageway such that the functional lumen can be pressurized independently from the passageway.

The catheters of the present invention also allow for catheter construction custom-designed without the need to compromise performance features. Such catheter constructions are possible by being able to customize the properties and materials of any given section of the catheter. Such customized properties include but are not limited to durometer, torque control, flexibility, axial strength, stiffness, etc. The present disclosure also includes variations of improved catheters that have gradual or customized transition sections that can be configured selectively. For example, any section of a polymeric tubing (and therefore a finished catheter construction) can include polymers having a low durometer, a moderate durometer, and a high durometer in the same region. The ability to improve transitions is just one example of the benefit of an improved catheter constructed in accordance with the teachings herein.

For purposes of explaining the features of the present invention, the polymeric strands/components represent the material sections described herein prior to being formed into a tubular wall. As noted herein, in some variations, a material section can be formed from a first polymeric material and extend in a spiral pattern. At some point, the first polymeric material terminates at an end and is joined to an end of a second polymeric material, which still extends or continues in the spiral pattern of the material section. In such a case, the material section is considered to have two different polymeric materials at different lengthwise regions. In additional variations, a material section comprises a polymeric material and extends spirally for a lengthwise region of the tubing, then terminates such that adjacent material sections join together to maintain continuity of the wall of the resulting tubing. It is also noted that, when referring to the joined construction of individual strands, the term tubular wall, polymer tubing, polymer layer, composite tubing, composite layer, etc., can include material sections comprised of one or more materials: metal, stainless steel, alloy, liquid crystal polymer (LCP), fibers, composite material or other similar structures.

It is noted that a transition section shall be used to describe the changing of one or more material strands with a different material. The term transition region shall describe the overall effect of the one or more transition sections. In some variations, the transition region does not include any transition sections because a material simply terminates. Therefore, catheter constructions of the present disclosure can have a transition region that gradually changes material properties over an axial length, or alternatively, the transition region can be a region of an abrupt change in material properties.

The present disclosure includes a number of variations of catheters having outer tubing layers formed from a plurality of materials to customize characteristics of the lengthwise regions of the catheters. Specific variations of catheters can also include this composite polymeric layer as being on an interior layer of the catheter construction, but in many variations, the custom composite layer is on the outer layer.

### BRIEF DESCRIPTION OF THE FIGURES

FIG. 1A illustrates a common design for a conventional balloon catheter.

FIGS. 1B and 1C illustrate examples of cross-sectional views of common balloon catheters.

FIG. 1D shows a conventional catheter having multiple durometer regions.

FIGS. 2A and 2B show an illustration of an improved balloon catheter incorporating a composite layer.

FIGS. 3A and 3B show partial cross-sectional views of improved catheters and also show a functional lumen extending within a material section of the composite layer.

FIGS. 4A to 4F show additional variations of an improved catheter having a spiral balloon.

FIGS. 5A to 5C illustrate cross-sectional views of a variety of composite layers to demonstrate some examples of lumens within material sections of the composite layer.

FIG. 6A illustrates additional variations of a device having a composite tube formed from a plurality of tubular extensions.

FIGS. 6B and 6C illustrate some examples of tubular extensions.

FIGS. 7A and 7B illustrate one example of the fabrication process to construct a catheter section under the present disclosure.

FIG. 7C illustrates a catheter section comprising a plurality of discrete polymeric strands of material that are wrapped about a mandrel or tube.

FIG. 7D illustrates an image of an example of a wrapping process.

FIG. 7E illustrates a configuration where polymeric strands are secured together prior to being helically wound.

FIG. 7F illustrates three additional variations of polymer strands arranged having varying properties.

FIGS. 7G and 7H show additional variations of polymer strands joined together end-to-end and lengthwise prior to being helically wound and formed into a tubular body.

FIGS. 7I and 7J depict additional variations of non-uniform strands joined together prior to forming a tubing for use in a catheter construction.

FIG. 8A illustrates another variation of a group of joined strands prior to forming the tubular section depicted in FIG. 8B.

FIG. 8B illustrates a catheter section formed from the strands shown in FIG. 8A.

FIGS. 9A to 9C show variations of strands having reinforcing structures.

FIGS. 10A and 10B show a picture of a plurality of strands extending next to a scale to illustrate a perspective of the strands for one variation of the catheter construction.

FIGS. 11A and 11B show two examples of sections of catheters having an outer layer that can be incorporated into a catheter or used as a stand-alone device.

FIGS. 12A to 12D illustrate another variation of the device that changes polymers incrementally to construct a wound catheter with a gradually changing transition region between different sections of the finished polymer tube.

FIGS. 13A and 13B show a plurality of material sections having additional discrete materials formed therein.

FIGS. 14A to 14C illustrate another variation of a composite polymer tube having a plurality of material sections where the material section is embedded within a polymer tube.

FIG. 15 shows a number of non-exhaustive design configurations to produce a hybrid region.

FIGS. 16A to 16F illustrate additional examples of various constructions of tube members for use with the devices described herein.

FIGS. 17A and 17B illustrate another example of customizing material sections using a transition material section that reduces in width.

FIG. 18A shows a traditional catheter construction where the polymer chains are aligned with an axis or axial length of the tube.

FIGS. 18B to 18E illustrate additional design variations for use in composite tubing sections having a high modulus of elasticity/stiffness.

FIGS. 19A to 19D illustrate another variation of a composite tube.

FIG. 20A to 20F illustrates another variation of fabrication of a composite tube.

FIG. 21 illustrates another variation of a composite tube that is formed with non-overlapping material sections.

FIG. 22 illustrates another variation of a composite tubing.

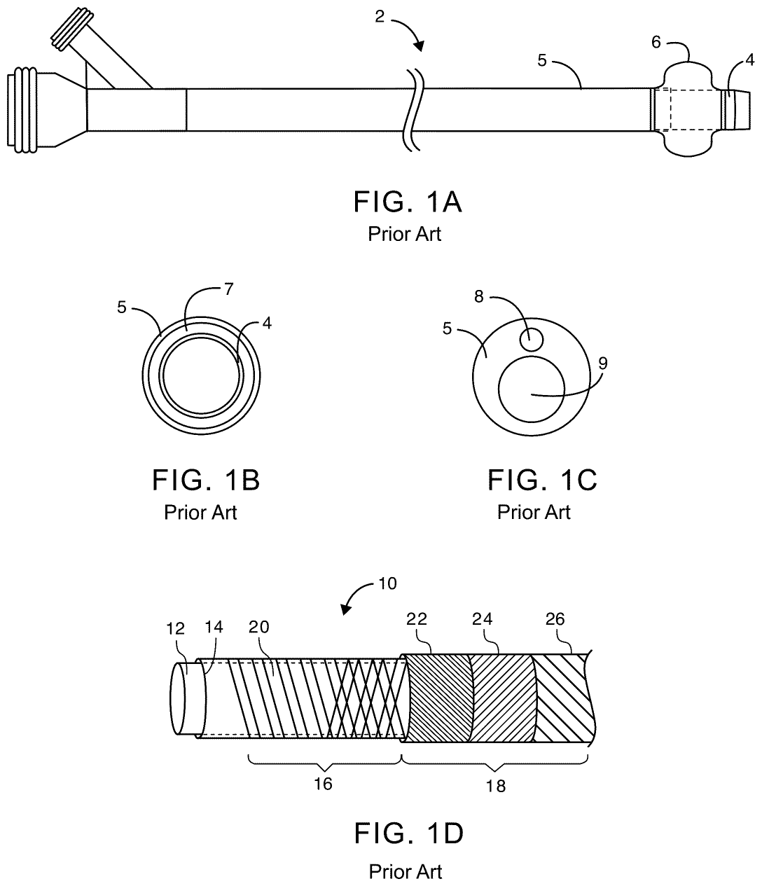

### DETAILED DESCRIPTION

The catheter configurations discussed herein can be used in a variety of devices where different regions are selected for customized properties and where a composite wall of the device carries at least one functional lumen that is suitable for fluid delivery. The configurations described herein can be incorporated into various medical devices or can be used as catheter shafts. Furthermore, in some variations, the construction features of the present disclosure are not limited to in-dwelling medical devices and can be used for any device requiring tubing.

The polymeric tubing described herein can be constructed in any manner that allows the material section configurations (and hybrid-regions) disclosed below. Such manufacturing means include but are not limited to: forming the polymeric tube by winding directly onto catheter shaft; forming the strands into composite sheets and then winding the sheet onto a structure to complete a catheter shaft; and/or first winding ribbons/strands onto a mandrel or support structure, then fusing the material into a tube, then transferring onto a catheter assembly.

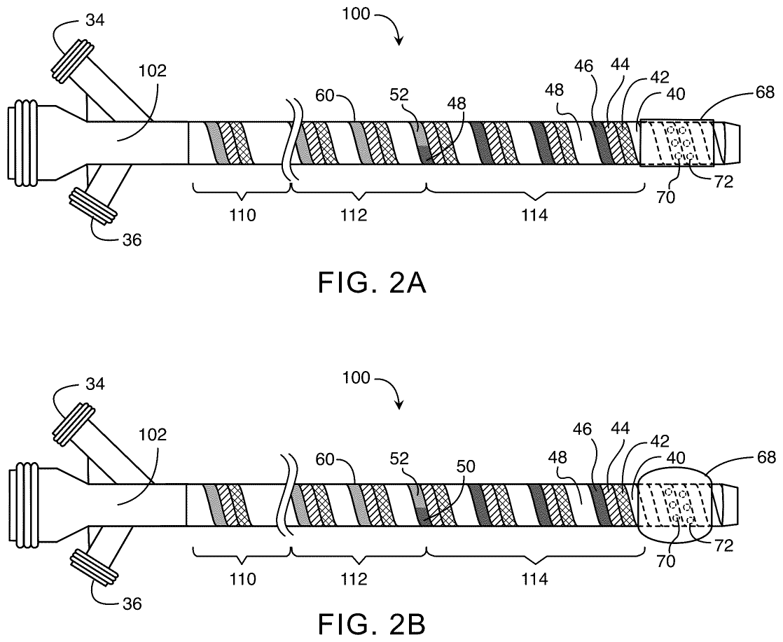

FIGS. 2A and 2B show an illustration of an improved balloon catheter 100 incorporating a composite layer 60, as discussed herein. Where the composite layer 130 comprises a plurality of material sections 40, 42, 44, 46, 48, 52 that extend spirally to form at least a portion of a wall of a shaft of the balloon catheter 100 where the material sections can each have unique structural properties such that each region 110, 112, 114 comprises unique structural characteristics that aid in navigation of the balloon catheter 100 through the desired anatomy. As shown in FIGS. 2A and 2B, the properties of a material section can change. For example, at the intersection of regions 112 and 114, the material section changes. This change can allow custom structural properties of the catheter tubing 60 in different regions.

Typically, the balloon catheter 100 includes a hub 102 with one or more fittings 34 36 that allow coupling of the balloon catheter 100 to an inflation source and/or negative pressure source. FIGS. 2A and 2B also show the catheter 100 having a flexible film attached to the composite shaft 60 for forming a balloon 68. The balloon 68 is adjacent to one or more ports 70 72, which are fluid outlets of functional lumens that extend through one or more of the material sections. FIG. 2B illustrates the film when pressurized by one or more of the ports 70. In an additional variation, the device 100 can include inflation ports 70 and vacuum ports 72, which allows pulling of a vacuum within the balloon 68. The balloon material 68 can comprise an elastic material or a non-elastic material such that the balloon is either distensible or non-distensible.

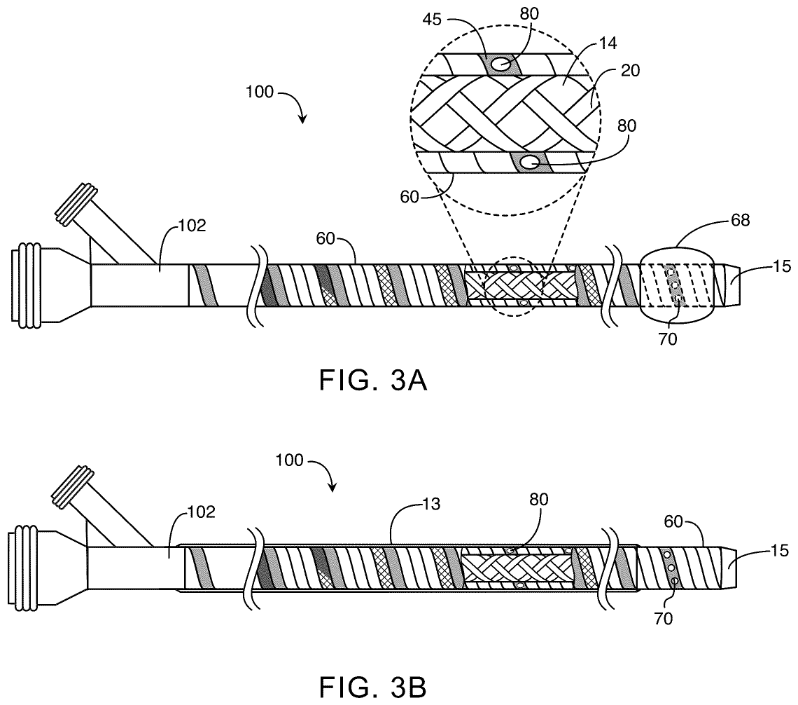

FIGS. 3A and 3B show a partial sectional view of an improved catheter 100 incorporating a composite outer layer 60 as discussed herein. The catheter construction discussed herein can incorporate any number of features known by those skilled in the art of catheter construction. Such features are omitted herein so that the focus of the improved catheter composite layer 60 can be explained. Furthermore, the improved catheter construction disclosed herein can be incorporated into any number of catheters that can benefit from customization of features provided by the improved polymeric layer 60. For example, such catheters include, but are not limited to, distal access catheters, sheaths, guide catheters, balloon catheters, intracranial support catheters, micro catheters, arterial line catheters, central venous catheters, pulmonary artery catheters, coronary and cardiac catheters, and peripheral catheters, etc.

As shown in FIG. 3A, in one variation of the device, the tubular construction or composite layer 60 of the catheter 100 extends from a hub and can overlay a braid 20, coil, or other support structure commonly used with catheters. The braid 20 is positioned about a tubular inner liner 14 (commonly constructed from PTFE, but other materials are within the scope of this disclosure). The liner can be interior to the support or braid 20. Alternatively, the support or braid 20 can be embedded within the liner 14 or the composite layer 60. As shown in FIG. 3A, the improved composite layer 60, is the outermost component of the catheter tubing. As noted below, the improved composite layer 60 can include any number of lengthwise regions that are better suited to transmit torque through the catheter 100 or to provide any additional structural feature to improve navigation and/or stability of the catheter. Positioning these polymeric torque transfer regions to make the composite layer for the catheter improves the effectiveness of the stability, flexibility, pushability, and/or torque transfer region as compared to conventional catheters that mainly rely on a braid 20 that is positioned within the catheter shaft. Additionally, the magnitude of combinations of the number regions in each catheter is nearly limitless. Therefore, while the catheters described herein may not show various differing regions, each catheter described herein can include any number of regions having different structural properties. In some variations, a catheter will not have any differing regions but only uses one or more material sections to provide a functional lumen to any portion of the catheter.

FIG. 3A illustrates a variation of a catheter device 100 similar to that shown in FIG. 2A, where the catheter 100 includes a distal tip 15 coupled to the end of the tubing 60. As shown, the distal tip 15 can comprise a soft polymeric material or other material. FIG. 3A also shows an expanded sectional view of the composite layer 60, where a material section 45 includes a fluid lumen 80 that extends through the material section 45. Accordingly, the fluid lumen 80 remains in a wall of the tube 60 and extends spirally with the material section 45. The fluid lumen 80 is typically fluidly coupled to an associated port on the hub 102 for coupling to a fluid or vacuum source.

FIG. 3B illustrates additional features that can be combined with any other variation of devices in the present disclosure. For example, the composite layer 60 can include an outer layer 13 positioned over all or a portion of the composite layer 60. The outer layer 13 can comprise a transparent or translucent material. In most cases, the performance and characteristics of the device 100 will be controlled by the selection of materials forming the composite layer 60, the incorporation of the braid/coil, or other support structure 20. In additional variations, the outer layer 13 typically will not affect the performance and/or characteristics of the device 100. FIG. 3B also shows a variation of a catheter 100 that includes one or more functional lumens extending through a material section in the composite wall 60 configured to function as a fluid delivery catheter 100 where openings 70 can deliver fluid or other substances through the functional lumen 80 that extends in any material section. It is noted that one or more openings 70 can be positioned along any section of the tube 60.

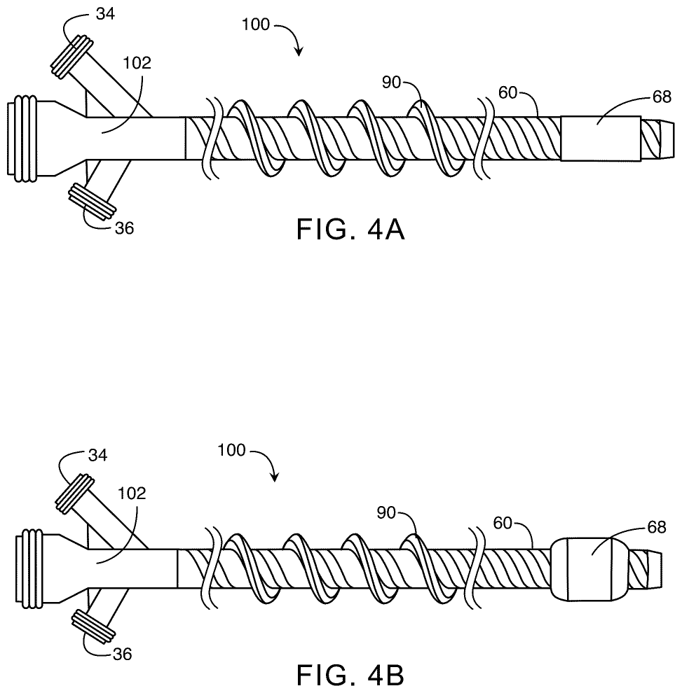

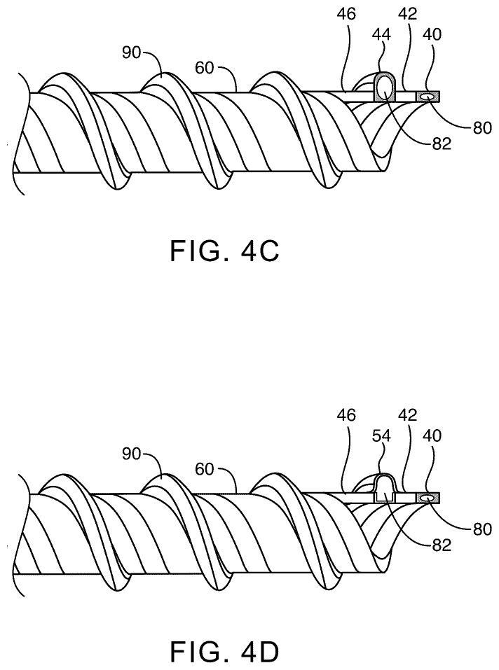

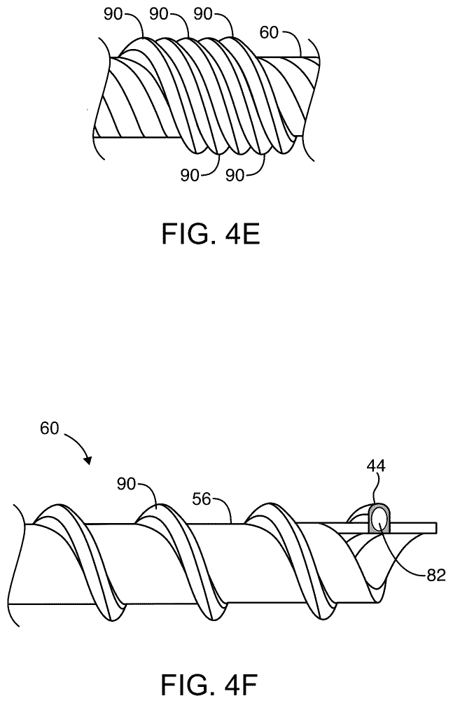

FIG. 4A illustrates yet another variation of an improved catheter 100. In this variation, one or more of the material sections 90 is configured with a functional lumen and is also configured to expand radially outward upon pressurization of the lumen 80 within the material section 90. As shown, this produces a spiral balloon 90 about the composite tube 60 that can be used to anchor the catheter device 100 within the vasculature. Moreover, because of the spiral configuration, the regions between the spiral balloon 90 permit blood flow through the vessel while balloon 90 is expanded and the device 100 is anchored. FIGS. 4A and 4B show a distal balloon 68 in reduced and expanded configurations, respectively. Each balloon 68, 90 can be fluidly coupled to separate inflation ports/openings 34, 36. In alternate variations, the balloons 68 90 can be coupled to a single inflation port. In addition, variations of the device 90 can include catheters that only have a spiral balloon 90 or a plurality of individual spiral balloons and optionally have fluid delivery openings as noted above.

FIG. 4C illustrates a partial view of a composite tube 60 with an expanded spiral balloon 90 and a cross-sectional view of a number of material sections 40, 42, 44, 46 of the composite tube 60 where material sections 40 and 44 include functional lumens 80, 82. As shown, material section 44 is configured for inflation into a spiral balloon about the composite layer 60. Lumen 80 can serve as a balloon inflation lumen or a fluid delivery lumen as discussed above. FIG. 4C also shows the material sections as having a rectangular cross-section. It is noted that the material sections can have different cross-sectional areas (e.g., material section 46 is wider than material sections 40 and 42). Additionally, the material sections can include different heights as discussed below.

FIG. 4D illustrates another partial view of a composite tube 60 with an expanded spiral balloon 90 and a cross-sectional view of a number of material sections 40, 42, 44, 46 of the composite tube 60 where material section 40 includes functional a lumen 80. However, in this variation the, balloon 90 is formed from a balloon material 54 attached to the exterior of the tube 60 such that the layer covers a cavity 82 or lumen. Pressurization of the lumen 82 causes the balloon material 54 to expand and form the spiral balloon 90. As shown, balloon material 54 is affixed in a spiral pattern about the lumen 82. Where the lumen 82 can serve as a balloon inflation lumen or a fluid delivery lumen, as discussed above. As noted above, the rectangular cross-sections are for purposes of illustrations. The material sections can have different cross-sectional areas. Additionally, the material sections can include different heights, as discussed below.

The spiral balloons discussed herein can be positioned at any portion of the device. For example, positioning the spiral balloon on a proximal or mid portion can allow the balloon to provide an anchor or stability. Positioning the spiral balloon on the distal portion can allow for anchoring or occlusion. For example, FIG. 4E illustrates the spacing of spiral balloons 90 to produce an occlusion region when expanded since there is no spacing between balloons 90. It is noted that this occlusion region can be formed by a single balloon 90 or multiple balloons. In most cases, and as shown in FIGS. 4A and 4B, the spacing between the turns of the spiral balloon will be sufficient to allow expansion but not occlusion of the vessel or organ in which the spiral balloon is located. However, in additional variations, the spacing of the turns of the spiral balloon can be sufficiently close to permit occlusion. In some variations, a single device can include a non-occlusive spiral balloon on one region and an occlusive spiral balloon on a second region. It is also noted that while the devices described herein included a distal balloon (e.g., 68 in FIGS. 4A and 4B) as well as one or more spiral balloons 90, variations allow for only a spiral balloon 90, only a distal balloon 68, a combination of both balloons or multiple spiral balloons and/or conventional balloons.

FIG. 4F illustrates another variation of a construction of a layer 60 having a spiral balloon structure 90 comprising a material section 44 with a lumen 82. However, in this variation, the material section 44 can be incorporated into a tube 56, having a spiral region/groove/channel removed to accommodate the material section 44. While the illustration shows a single spiral structure 90, any number of spiral structures can be fabricated into the tube 56. Moreover, the spiral structures 90 can comprise occlusion regions as noted above or can be spaced as shown. Additional variations

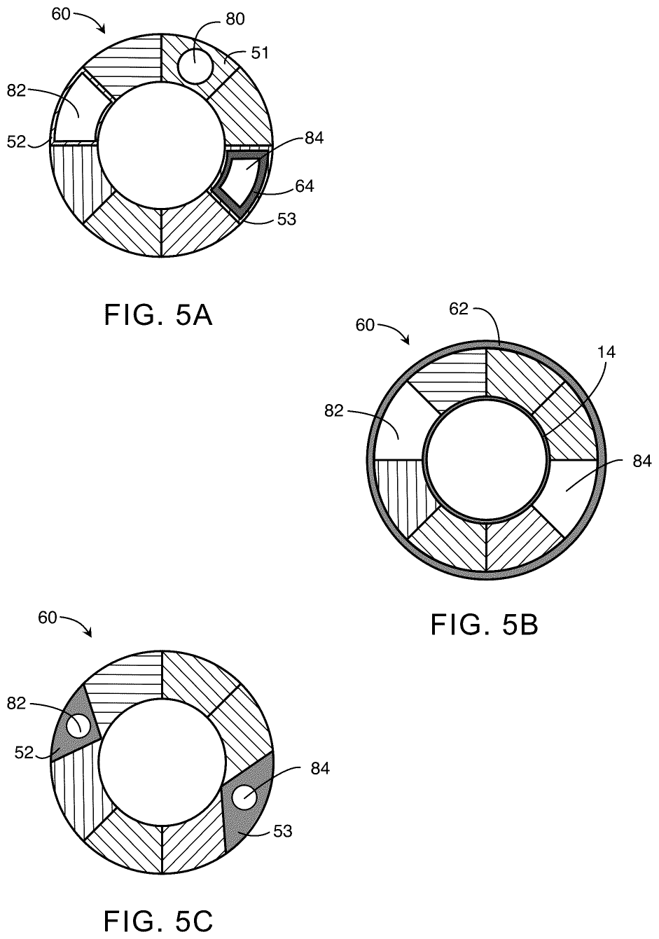

FIGS. 5A to 5C illustrate cross-sectional views of a variety of composite layers 60 to demonstrate some examples of lumens within material sections of the composite layer 60. FIG. 5A shows a composite layer 60 having lumens 80, 82, 84 within material sections 51, 52, 53. Lumen 80 represents a lumen that is machined or otherwise formed into material section 51. Alternatively, or in combination in the same composite layer 60, lumen 82 can be formed by hollowing out the material in the material section 52. In addition, the lumen 84 can include a braid/coil 64 positioned within the lumen 84 and/or material section 53. Alternatively, the braid/coil can be a reinforced polymer.

FIG. 5B illustrates an alternative design for a composite layer 60 that includes functional lumens 82, 84. In this variation, the composite tube 60 includes lumens 82 84 formed between adjacent material sections. For example, material sections can be removed from the tube 60 to provide space for lumens 82 84. Then, an exterior jacket can be fused over the composite tube 60 such that the region between the exterior layer 62 and the inner liner 14 forms lumens 82, 84. It is noted that FIGS. 5A and 5C do not show the composite layer 60 as having any inner tubular structure 14 for purposes of illustration. Any variation of the composite tube 60 can include an inner liner. Alternatively, the inner liner can be omitted from the construction. In additional variations, the exterior layer 62 can cover the full circumference as shown or can just cover the lumens 82, 84.

FIG. 5C illustrates a composite layer 60 where material sections 52, 53 comprise different cross-sectional shapes than the remaining material sections in order to maximize and/or accommodate the associated lumens 82, 84.

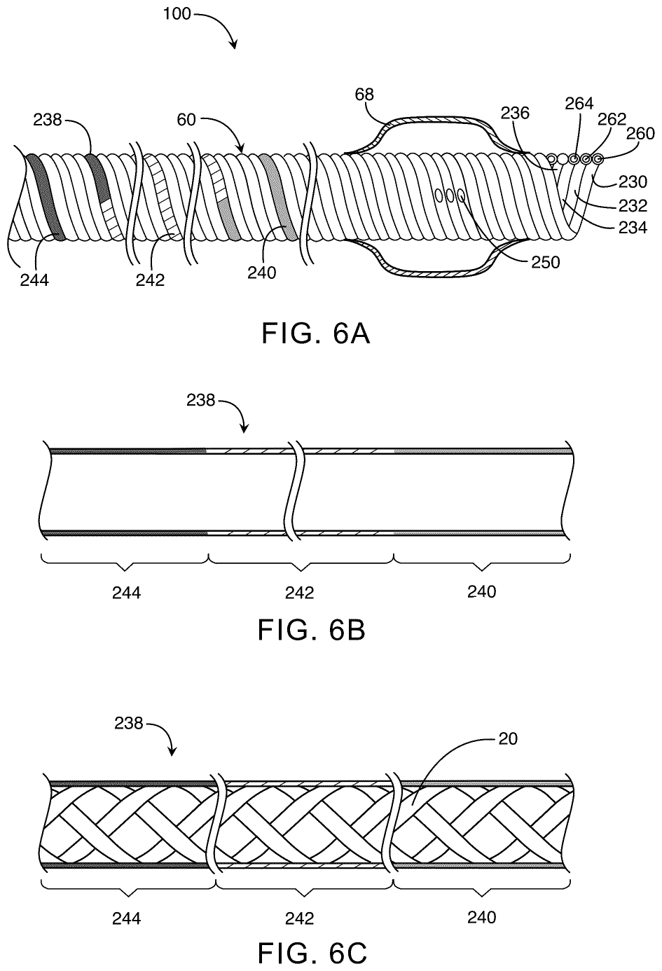

FIGS. 6A to 6C illustrate additional variations of a device 100 having a composite tube 100 with a balloon 68. As discussed herein, alternate variations of the device 100 can include fluid delivery openings in place of or in addition to a balloon 68. The device 100 shown in FIG. 6A comprises a plurality of material sections comprising a plurality of tubular extensions (e.g., microcatheters) 230, 232, 234, 236, 238 joined together to form the composite tube 100. It is noted that the tubular extensions 230, 232, 234, 236, 238 can be used with the other shapes of material sections disclosed herein.

FIG. 6A also shows a composite tube 60 having tubular extensions 230, 232, 234, each having a lumen 260, 262, 264. Tubular extension 236 is illustrated as being solid. However, variations of the devices 100 described herein can include each tubular having a lumen, one tubular extension having a lumen, or any combination of tubular extensions having lumens. The tubular extensions can include one or more openings 250. As noted above, one or more openings 250 can be used to pressurize the balloon, while one or more openings can be used to depressurize the balloon. Alternatively, the openings can be used to convey or remove fluid from the body when they are positioned outside of the balloon or used in a device not having a balloon.

FIG. 6A also shows a tubular extension 238 structural properties that change over the length of the extension 238. For purposes of illustration, tubular extension 238 shows three different structural properties 240, 242, 244. In one example, regions 240, 242, 244 represent changing durometers of the tubular extension 238 from a hard durometer 244, to a medium durometer 242, to a soft durometer 240. As noted above, any number of structural properties can be changed with the designs disclosed herein. In addition, one or more of the tubular extensions can have different structural properties as discussed herein. In addition, the tubular extensions in a section or entirety of the device 100 can having the same structure properties, but one or more of the extensions will be visually distinguishable from the remaining extensions. Although not shown, one or more of the tubing extensions can be configured to expand radially outward from the composite tube 60 as discussed above.

FIG. 6B shows a partial cross-sectional view of a tubular extension 238 comprising three different structural properties 240, 242, 244 that change over a length of the extension 238. FIG. 6C shows another variation of a tubular extension 238 that changes structural properties 240, 242, 244 over a length of the extension 238, and the extension also includes a braid, coil, or other support structure 20.

Additional variations of the improved construction can be used in any polymeric tubular structure. It should be noted that any catheter construction or polymer tubing disclosed herein is not limited to a single uniform outer diameter across the entire catheter. As disclosed below, the catheters and polymer tubing of the present disclosure can have an undulating outer diameter. Alternatively, or in combination, the outer diameter can vary throughout various lengthwise regions of the catheter. The term lengthwise region is intended to mean a region of any length along an axis 105 of the tube construction. The catheter constructions and tubular constructions disclosed herein can have any number of conventional cross-sectional shapes. For example, variations of the devices can include catheters that have different diameters and/or cross-sectional shapes at different regions. Some sections of catheters and tubular constructions can include round cross-sectional shapes that change to non-round shapes.

The following discussion represents some variations of joining material sections into a composite layer. Any variation described below can include one or more functional lumens as described above.

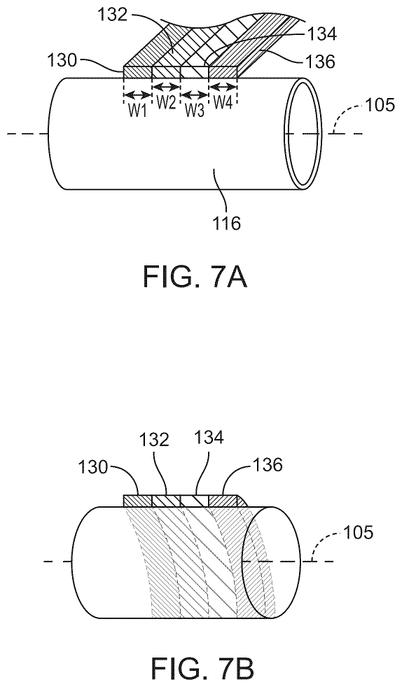

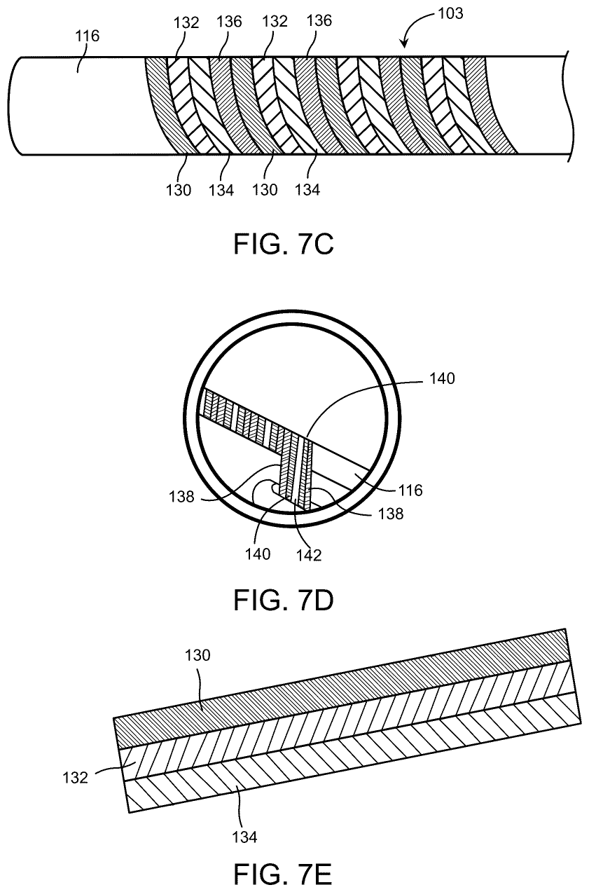

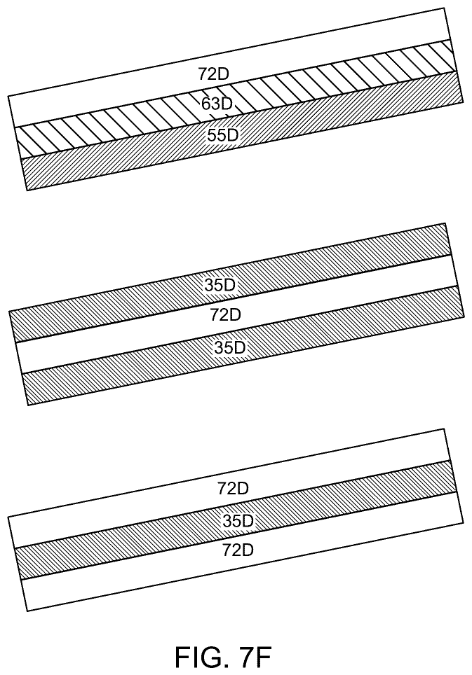

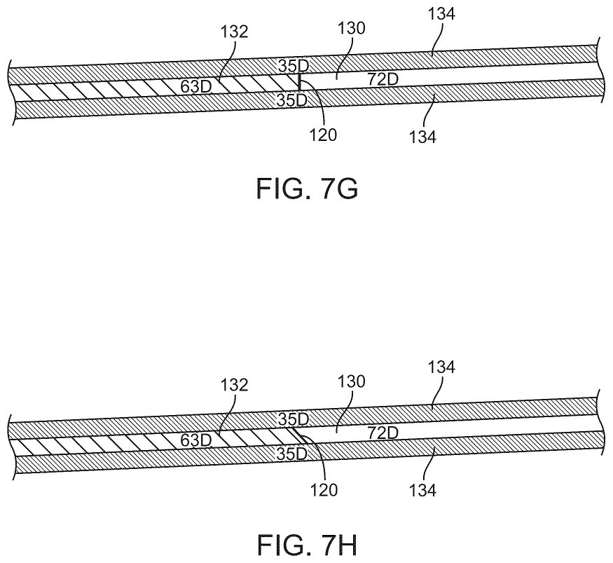

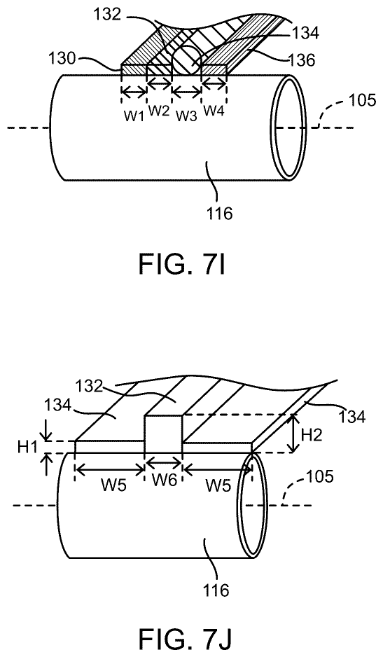

FIGS. 7A and 7B illustrate an example of the fabrication process to construct a catheter section under the present disclosure. It is contemplated that any manufacturing process that creates a catheter or catheter layer with a plurality of material sections is within the scope of this disclosure. For example, such manufacturing processes can include wrapping polymer strands (as shown), 3D printing, extrusion, etc.). As shown in FIG. 7A, a number of polymer strands or ribbons are placed in a pattern to coincide with material sections 130, 132, 134, 136 and can be wrapped about a structure 116. The structure can comprise a mandrel, tube, or a braid/liner of a catheter structure. Once the polymeric strands are wrapped, they are fused or otherwise joined together to form a layer as described here (e.g., see layer 103 of FIG. 2A). In one variation, the wrapped and joined polymer ribbons form a wall layer of a catheter after they are fused together. Alternatively, the polymer ribbons can form an outer layer over a tube, braid, and/or coil 116 and form a portion of a catheter section. For the sake of convenience, the polymer strands/ribbons/extrusions shall be referred to as polymer strands. The present invention includes the polymer sections as having any shape necessary to complete the catheter section. As shown, a cross-section of the polymer strands can be rectangular. Alternatively, the polymer strands can be oval, round, or have any other shape. In additional variations, polymer strands of different shapes and sizes can be combined to form a layer. Moreover, the polymer strands can comprise single-lumen extrusions/tubes that are collapsed and melted/fused down. Alternatively, the strands can be extruded or otherwise manufactured to be solid. In another variation, the lumen of each polymeric strand is left intact. In a typical variation, the strands are wound over a braid or coil (as discussed above). In an additional variation, the polymer strand construction discussed herein can be used to form an inner layer of a catheter (instead of or in addition to a polymeric liner), with a separate construction being used for an external layer of the catheter. In an additional variation, even though the disclosure herein discusses strands and material sections as comprising polymers. A strand or material section can comprise a non-polymeric material (e.g., metal, stainless steel, alloy, liquid crystal polymer (LCP), fibers, composite material, or other similar structures). Strands can be different materials, shapes, sizes and mixed together, or can be placed and removed to leave voids. Strands can also be different materials, shapes, sizes and mixed together, or can be placed and removed to leave voids.

For purposes of explaining the features of the present invention, the polymeric strands/components represent the material sections described herein prior to being formed into a tubular wall. As noted herein, in some variations, a material section can be formed from a first polymeric material and extend in a spiral pattern. At some point, the first polymeric material terminates at an end and is joined to an end of a second polymeric material, which still extends or continues in the spiral pattern of the material section. In such a case, the material section is considered to have two different polymeric materials at different lengthwise regions. In additional variations, a material section comprises a polymeric material and extends spirally for a lengthwise region of the tubing, then terminates such that adjacent material sections join together to maintain continuity of the wall of the resulting tubing.

Regardless of the fabrication process, the polymer strands in each of the material sections 130-136 can comprise polymers of varying compositions. In one example, the polymers can be a common material (e.g., PEBAX) where each strand in a respective material section 130-136 comprises a different durometer. For example, the strands can have the following associated durometers: 130-72D, 132-63D, 134-35D, and 136-45D. Clearly, any number of variations are within the scope of this disclosure.

FIG. 7A also illustrates the plurality of material sections 130, 132, 134, 136, each having a respective width W1, W2, W3, and W4, measured along an axial length 105 of the tube. In this illustration, the axial length 105 is the axial length of the core or tube, which will generally be similar if not the same as an axial length of the finished tube or a catheter having a layer formed by material sections 130, 132, 134, 136. In the case of materials not yet formed into a tube structure, the width is measured in a plane that is perpendicular to a length of the strand. As shown in FIG. 7B, the material sections extend in a spiral direction along the axial length 105 to form a continuous wall as discussed herein.

FIG. 7C illustrates a wall section 103 after a plurality of discrete polymeric strands of material in material sections 130, 134, 132, 136 are joined together a support structure 116. Section 103 can be incorporated into a medical catheter, medical device, and/or other tubing.

FIG. 7D illustrates an image of an example of a wrapping process where strands of polymer form material sections 138, 140, 142 and are wrapped directly onto a catheter reinforcement braid 116. (Alternatively, the strands could be wrapped onto a mandrel, fused or partially fused together, and then transferred onto the catheter braid as conventional catheter construction). In this variation, the strands 138-142 are separate and are wrapped such that the strands are in contact for joining to form a sealed connection between adjacent materials, such as by thermal fusing. However, any process that results in joining of adjacent materials can be used.

While the variations disclosed herein show a single layer of various material sections forming a wall of the tubing, it is noted that tubings can be formed from multiple layers where each layer comprises multiple material sections. Where each layer can have the same or different sequence of material sections.

FIG. 7E illustrates a configuration where polymeric strands 130-134 that are secured together prior to being helically wound to form material sections 130-134. For example, the strands can be fused together or tacked together prior to winding.

FIG. 3F illustrates three additional variations of polymer strands arranged having varying properties. In the illustrated example, the durometer of the strand is shown. However, the polymer strands can vary other properties as needed. As shown in the bottom two variations, two strands of a similar configuration can be placed adjacent to a dis-similar strand. When formed into a tubular member, the center material section will be bounded by material sections having the same polymer.

FIGS. 7G and 7H show additional variations of polymer strands 130, 132, 134, 130-134 joined together end-to-end and lengthwise prior to being formed into a wall where strands 134 will ultimately form the material sections on either side of the material section formed by strands 130 and 132. In this variation, strands 130 and 132 are joined end-to-end at a transition section 120 to allow for a transition of materials lengthwise along an axial direction of the finished catheter. This means that when formed into the tubular member/wall, the center material section comprises material 130 joined to material 132 at edge 120. The joint or transition section 120 between strands 130 and 132 can be an abrupt transition section 120, as shown in FIG. 7G or an angled or tapered transition section 120, as shown in FIG. 7H.

FIGS. 7I and 7J depict additional, but not exhaustive, variations of strands 130, 132, 134, 136 being joined together where the strands are not uniform. For example, FIG. 7I illustrates a strand 134 as having a circular cross-sectional shape. As noted above, any type of cross-sectional shape can be used. In such cases, a width W3 of the strand 134 can be considered its widest dimension along the axis. In some variations, the size of the strand 134 will cause the resulting material section to protrude slightly from a surface of the tube. FIG. 7J illustrates such a case, where a height H1 of certain strands 134 are joined with a strand 132 having a greater height H2. FIG. 7J also illustrates the widths W5 and W6 of the strands as not being uniform. Again, any permutation of shapes, sizes, widths, heights, etc., can be combined to make a polymer layer. It should be noted that any strand of material incorporated into a composite polymeric layer can include strands of material having a melt temperature different than one or more adjacent strands. It is also noted that in some variations, one or more strands can be non-meltable (i.e., a thermoset material, or metals, Teflon, etc.) that are mechanically held by the adjacent strands but do not melt. In additional variations, the non-meltable strand is used during formation of the tubing and then removed to create a void or pattern.

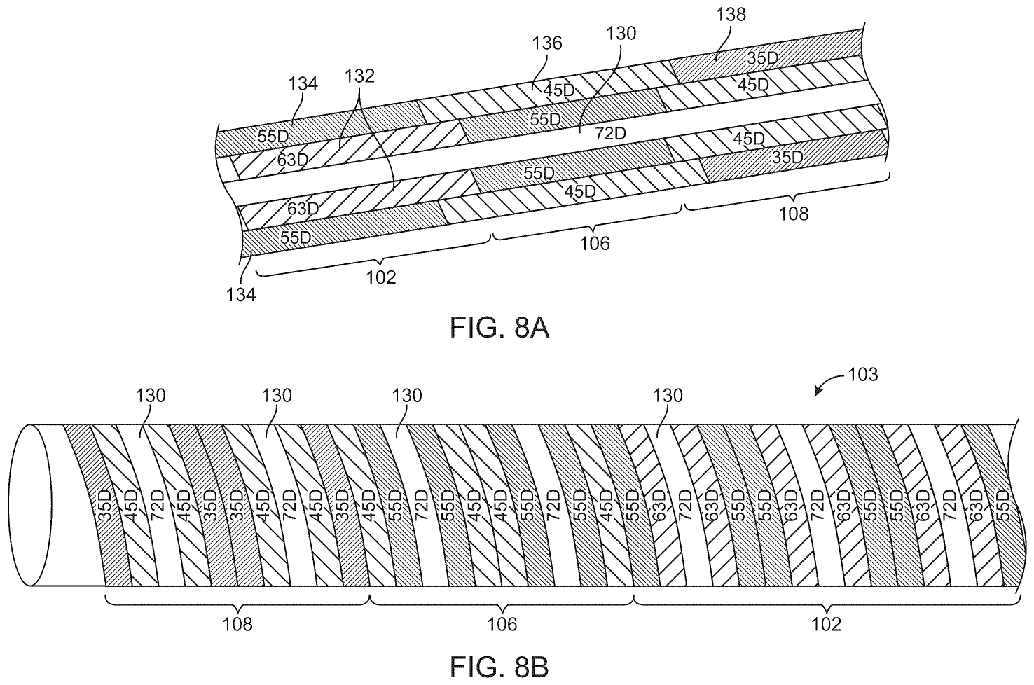

FIG. 8A illustrates another variation of a group of joined strands 130-138 prior to forming the tubular section depicted in FIG. 8B. As shown, the strands comprise different properties, resulting in different sections 102, 106, and 108 for the catheter. When wound, as shown in FIG. 8B, the varying of the composition of material sections 130-138 form different axial sections 102, 106, 108 extending lengthwise along the tubular layer 103. In both variations depicted in FIG. 8A and FIG. 8B, the strands/tubular layer 103 include a single strand 130 that will extend continuously as a material section 130 over a full length of the finished tube 103. In this example, the strand 130 comprises a 72D material and can be ultimately used as reinforcement for the finished catheter. (mainly used to transmit torque and provide stability through a normally soft and flexible distal region that usually does not transmit torque well and usually has poor stability).

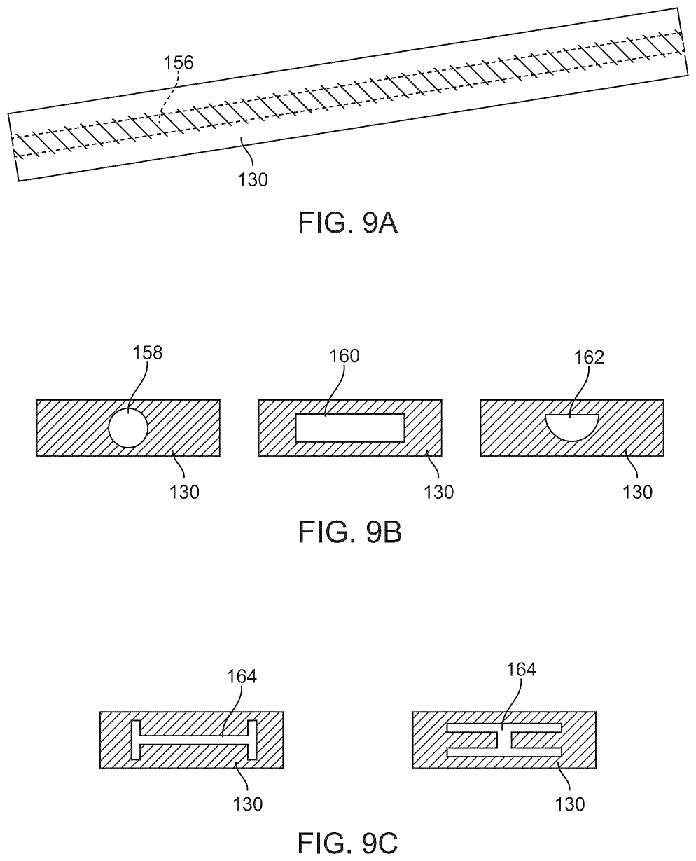

FIG. 9A illustrates an additional variation of a catheter construction described herein where a polymer strand 130 includes a support member 156 extending therethrough that reinforces the strand 130 or provides alternate structural and characteristics. The support member 156 can extend through a full length of the strand 130 or partially through a strand. Moreover, a variation of the reinforced strand 130 can include a plurality of support members that extend through a strand. FIG. 9B illustrates a cross-sectional view of a strand 130 to illustrate some cross-sectional shapes of reinforcement members. As shown, the reinforcement member can have circular 158 or elliptical cross-section, the support member can have a rectangular or square 160 cross-section, or the support member can comprise a D-shaped 162 cross-section. The support member can comprise a metal, an alloy, or polymer. For example, the support member can comprise SS wire, a shape memory wire, a drawn-filled tube, or a composite fiber material. It can be in a cable, braid, coil, strand, etc., or any shape/structure/material used to provide support. FIG. 9C illustrates various complex cross-sectional shapes 164 for a support member within a strand 130. In certain variations, the catheter section can comprise different cross-sectional shapes in different sections of the catheter. For example, it was found that strands with a circular or oval cross-sectional shape are better suited for a distal region of a catheter while strands with a D-shaped support member are useful at the mid or proximal region of the catheter.

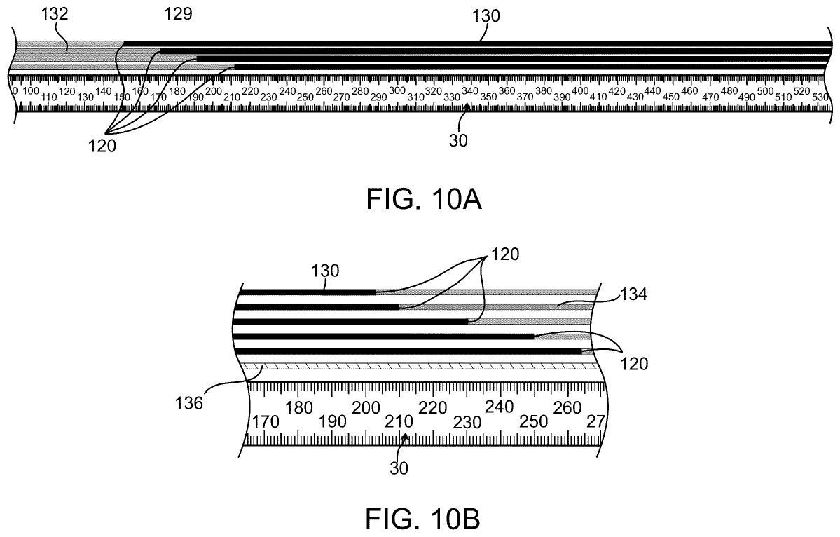

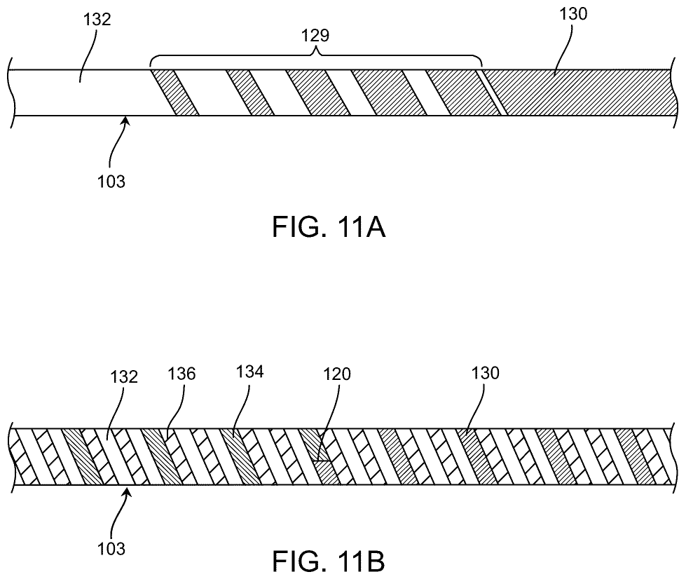

FIGS. 10A and 10B show an example of strands 130 and 132 extending next to a scale 30 to illustrate a perspective of one example of strands 130 and 132 that ultimately form a tubular member as described above where the overlap or staggering of the polymer end-joint locations, produces a finished polymer tube/catheter construction with a transition region 129 that is significantly improved over conventional catheter constructions. FIGS. 10A and 10B demonstrate how the overlap or staggering of the polymers at individual transition sections 120 (where the materials each have a butt-joint location) such that the end of 130 adjoins the end of 134), and will when wrapped, result in a significantly improved transition region 120 over the conventional catheters discussed above. As shown, the configuration of FIG. 10A includes staggered transition section 120, which creates a transition region 129 similar to that shown FIG. 11A. As noted herein, when strands 130 and 132 are formed into a tubular member, the strands 130 collectively form a material section that changes from a first material over region 129 to a second material having the material of strands 132. FIG. 10B shows a variation similar to the example of FIG. 10A with strands 134 joined/spliced end-to-end with strands 130. However, strand 136 remains continuous. When fabricated into a tubular member, strands 134 form a material section that changes in materials as described with respect to FIG. 10A, but the tube section is formed by FIG. 10B includes a material section formed by strand 136 that remains constant.

FIGS. 11A and 11B show two examples of sections of catheters having an outer layer 103 that can be incorporated on a catheter or used as a stand-alone device/structure. FIG. 11A illustrates a material section 130 formed from a first polymer and a material section 132 formed from a second polymer. The outer layer 103 includes a lengthwise region 129 of the tubular layer where a width of the first material section 130 and a width of the second material section 129 both change in width along the lengthwise region 129, causing a structural property to change over the first lengthwise region 129. As shown, the right side of FIG. 11A comprises a tubular member where an entirety is formed from material section 130 and a left side where an entirety of the material section 132 is formed from material section 132. In the transition region 129, the widths of the respective material sections inversely change along the lengthwise region 129 such that as the width of the first material 130 decreases towards the left and the width of the second material section increases. These transition regions can be made as long and gradual as desired by adjusting the length of section 129 and by adjusting the number of strands/ribbons used to give drastically improved and superior transition regions compared to conventional catheters.

FIG. 11B illustrates a variation of a tubing 103 having a plurality of material sections 130, 132, 136 spirally wound to form the tubing 103 where the tubing 103 includes a joint 120 where material section 130 changes to a different material 134, that continues in the spiral pattern of material 130. This end-to-end joining of materials allows the material section to continue while changing materials.

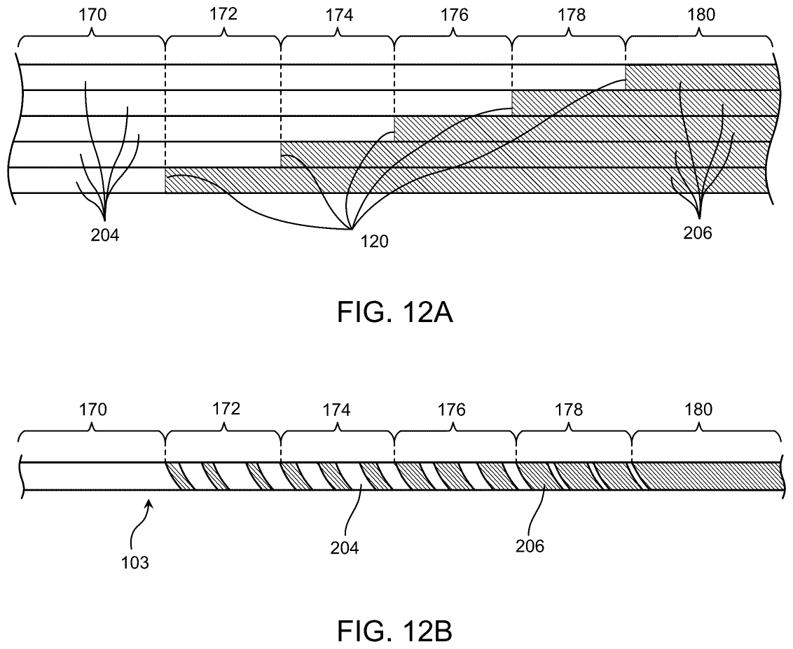

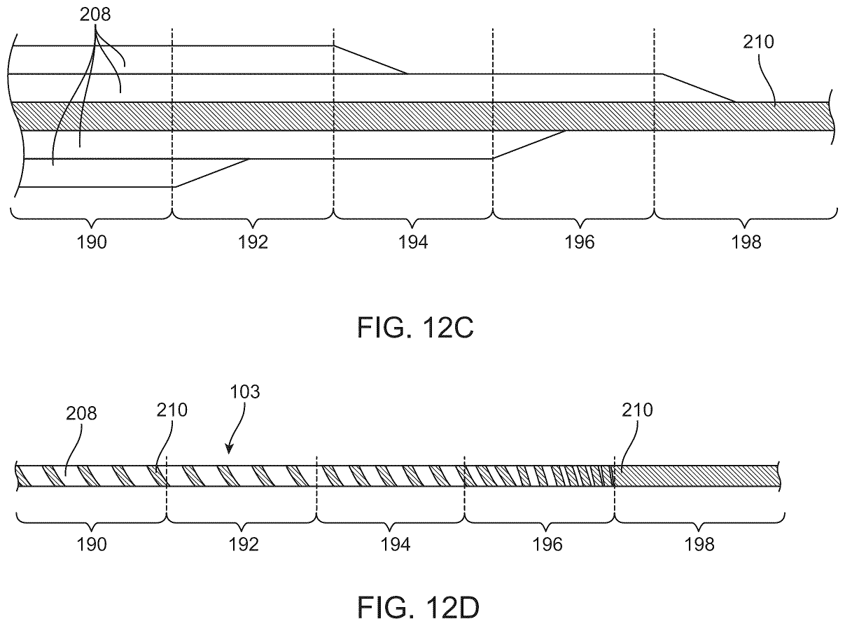

FIGS. 12A to 12D illustrate another example of an arrangement of strands to form a tubular member for use in a catheter. FIGS. 12A and 12C show a group of joined strands that can be varied to produce configurations as shown in FIGS. 12B and 12D, respectively. FIG. 12A illustrates a 5-strand construction, where one end of the joined strands comprises strands 204 of a first polymer. The strands 204 of the first polymer are each replaced at individual transition sections 120 that are staggered to gradually replace strands 204 with strands 206 of a second polymer over a transition region comprising length 172, 174, 176, and 178. This construction allows for a gradual variation over the transition region 172, 174, 176, 178 along the finished tubular assembly 103 (as shown in FIG. 12B) that has the properties of the first polymer in a first lengthwise region 170 and gradually changes over transition region 172, 174, 176, and 178 to the properties of the second polymer until lengthwise region 180 comprises all of the second polymer. The transitioning of materials in lengthwise regions 172, 174, 176, 178 represents an example of gradually transitioning material properties over a lengthwise transition region of a tubular assembly 103 or finished catheter construction. Clearly, any number of material sections or widths of material sections can be used to increase or decrease the rate of transitioning material properties. Moreover, variations of the devices described herein do not require staggering of transition sections 120. While staggering is usually desired to obtain a gradual transition, the transition regions can comprise an abrupt change in materials when desired.

It is noted that a transition section shall be used to describe the changing of one or more material strands with a different material. The term transition region shall describe the overall effect of the one or more transition sections. In some variations, the transition region does not include any transition sections because a material simply terminates. Therefore, catheter constructions of the present disclosure can have a transition region that gradually changes material properties over an axial length, or alternatively, the transition region can be a region of an abrupt change in material properties.

FIG. 12B also illustrates that each lengthwise region 172, 174, 176, 178 comprises at least two material sections 204 and 206, where a width of a material section 204 or 206 increases or decreases while the other material section 206 or 204 decreases or increases respectively. The variation of the tube 103 is shown in FIG. 12B also includes lengthwise regions 170 and 180 entirely formed a single material section. Again, any tube construction 103 discussed herein can be incorporated into a catheter construction as shown in FIG. 2A, or such tube construction 103 can be incorporated into any medical device or non-medical device.

As shown, the catheter section can comprise the various sections: section 170 consists of 5 strands of a first polymer (5 and 0); section 172 consists of 4 strands of the first polymer and 1 strand of the second polymer (4 and 1); section 174 comprises 3 strands of the first polymer and 2 strands of the second polymer (3 and 2); section 176 comprises 2 strands of the first polymer and 3 strands of the second polymer (2 and 3); section 178 comprises 1 strand of the first polymer and 4 strands of the second polymer (1 and 4); and section 180 comprises 5 strands of the second polymer (0 and 5). The construction of FIG. 12A produces the catheter shown in FIG. 12B after the strands are helically formed and melted into a catheter section.

FIG. 12C illustrates a plurality of joined strands where section 190 comprises 4 strands 208 of a first polymer and a single strand 210 of a second polymer (4 and 1). As shown, in the change to region 192, one strand 208 is tapered, leaving only four strands (3 and 1). The next section, 194 another strand, 208, is tapered, leaving only 3 strands (2 and 1). The process continues through sections 196 (1 and 1) until the strand 210 of the second polymer remains. The wrapping of the joined strands is adjusted (e.g., the pitch is altered) such that the reduction in number of strands does not leave any openings or gaps between strands. This construction produces a tube construction 103 similar to FIG. 12D As shown, the tubular construction 103 includes two material sections in lengthwise region 109. The width of the material section 210 increases in section 192 relative to section 190, while the width of material section 208 decreases in section 192 relative to section 190. The widths of material sections 208 and 210 continue to inversely change through lengthwise regions 194 and 196 until region 198 includes a single material section 210. The construction shown in FIG. 12D shows a tubular section 103 having transition regions 192, 294, 196 where the material sections change, but there are no transition sections of materials 208 since the material just terminates, as shown in FIG. 12C. While the construction of FIGS. 12A/12B and 12C/12D are different; both designs produce a shaft that transitions from a first material property to a second material property using a very gradual basis. This graduation and uniformity are significantly greater than what can be produced with conventional catheter technology. One example of the material properties is stiffness/softness. For example, the catheters of 12B and 12D can transition from a relatively stiff material property at, e.g., 170 of FIG. 12C and 190 of FIG. 12D to a much softer material property at, e.g., 180 of FIG. 12B and 198 of FIG. 12D. The transition region (e.g., 172-178 FIG. 12B and 192-196 of 12D) can be customized by selection of polymers, length of transitions, etc., to produce transitions that were simply not found in currently available commercial catheters. It is also noted that the lengths of regions 170-180 and 190-198 (as well as the lengths throughout this disclosure) are intended to convey the principles of the present designs. The lengths are not required to be the same and are not to scale unless otherwise claimed.

Clearly, the length of each section shown in FIGS. 12A and 12C is intended for illustrative purposes only. In addition, any number of polymer strands can be used along with any number of polymers. Moreover, it is noted that in FIG. 12A, a material section can be considered all of the separate elements 204 of the same material. Therefore, region 170 includes a material section that changes in width stepwise to region 172 and so forth. The change in width can be stepwise or incremental, as shown. Alternatively, the change can be tapered such that the change in width is continuous, as shown by regions in FIG. 12C where the ends of material 208 taper off.

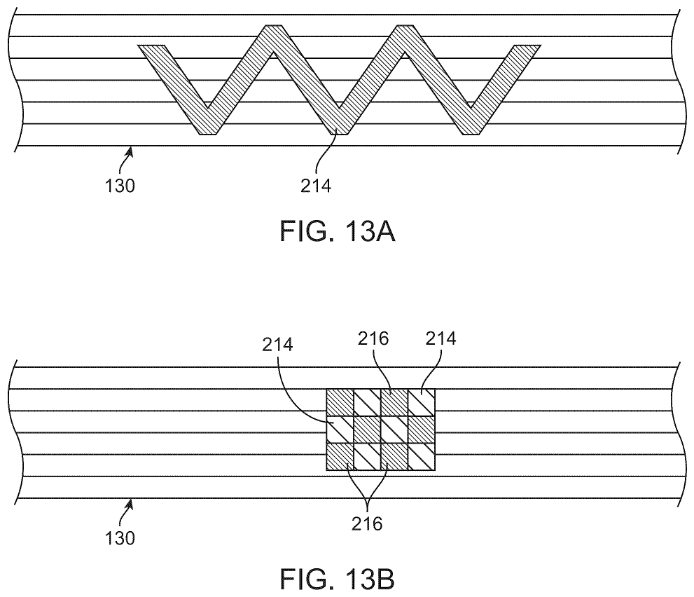

FIGS. 13A and 13B depict another feature of the catheter construction where a plurality of strands (either a similar polymer or different polymers) is joined together, as discussed above. However, in these variations, a variety of discrete materials (i.e., polymers, metals, composites, alloys, etc.) can be patterned on the joined strands 130. In FIG. 13A, a polymer is patterned into the illustrated shape 214. The base strand 130 can be removed, or the polymer 214 can be positioned on top of the base strands. Likewise, multiple polymers 214 and 216 can be positioned on a base strand 130 of polymers. In alternate variations, the base polymer strands 130 can be removed such that the patterned polymer 214 or 216 can be positioned in the space left by the removed base strand 130. The finished assembly 130 can be fabricated into a tube construction for incorporation as a catheter or other medical device shaft.

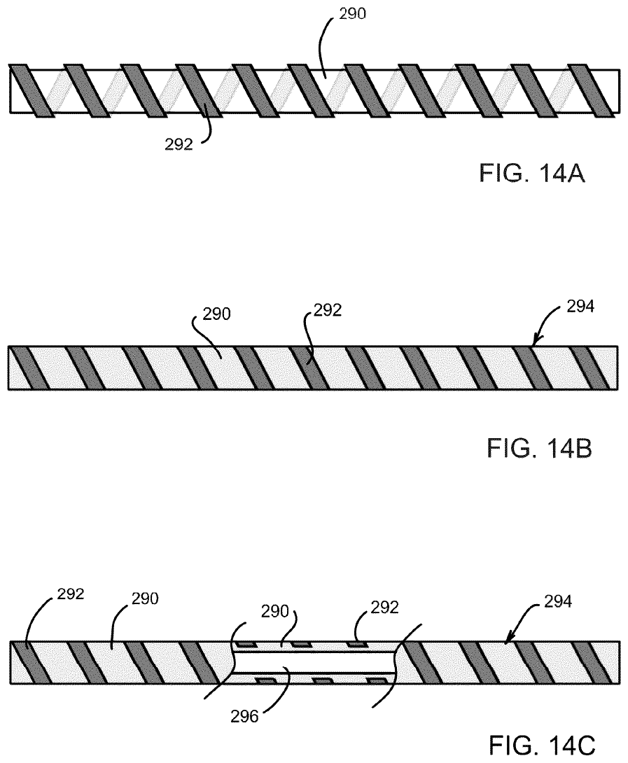

FIGS. 14A to 14C illustrate another variation of constructing a composite polymer tube 294 having a plurality of material sections under the present disclosure. As shown in FIG. 14A, the initial construction can comprise a conventional polymer tube 290 with one or more strands 292 wrapped about the tube 290. The tube 290 and strand 292 are then heat fused together to produce a composite polymer layer 294 where the strand 292 becomes at least partially embedded within the tube 290 such that the polymer layer 294 comprises a first material section comprising material 290 of the tube and a second material section comprising 292 of the strand. Clearly, any number of variations of strands (as described above) can be embedded within the tube. Moreover, the outer diameter of the polymer layer 294 can include undulations. FIG. 14C illustrates the polymer tube 294 with a portion removed to highlight the cross-sectional area of the polymer layer. In an additional variation, the construction of FIGS. 14A to 14C can replace the conventional polymer tube 290 with a composite polymer tube with varying material sections constructed as described herein.

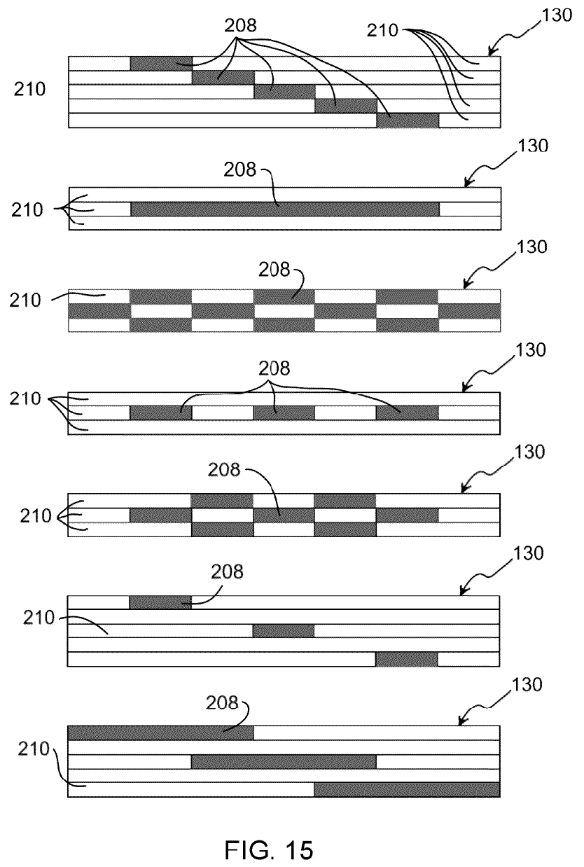

FIG. 15 shows a number of non-exhaustive design configurations to produce a hybrid region. The hybrid region of the catheter/finished tube is formed from a plurality of materials 130 joined together, where a base material 210 is interrupted by a discrete section of a second material 208 having different properties than the base. For example, in one variation of the design, material 210 can comprise a stiffer/harder durometer material or polymer, while material 208 comprises flexible/soft material or polymer. Clearly, any material properties other than hard/soft materials can be selected and configured into a hybrid region.

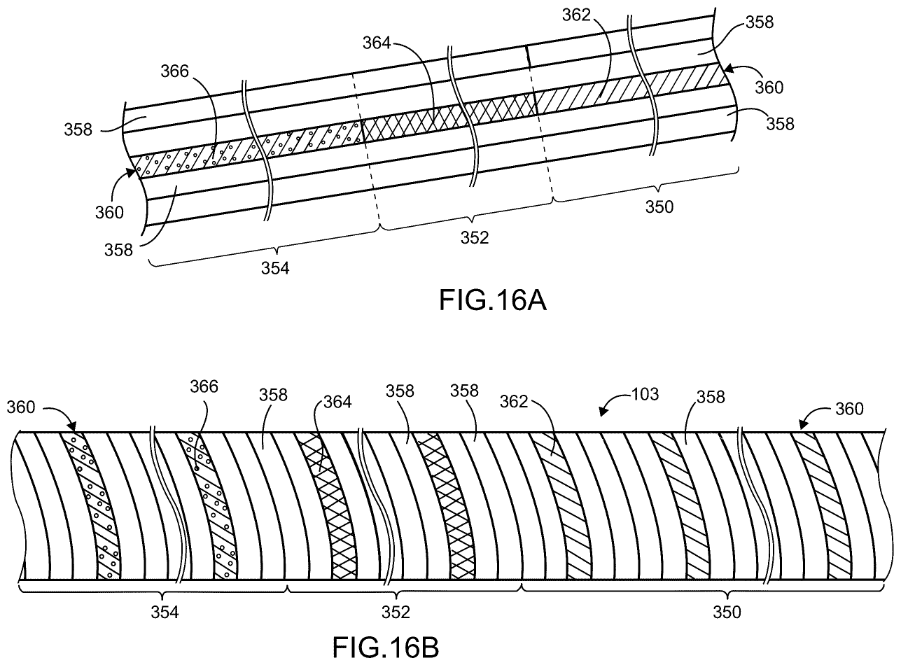

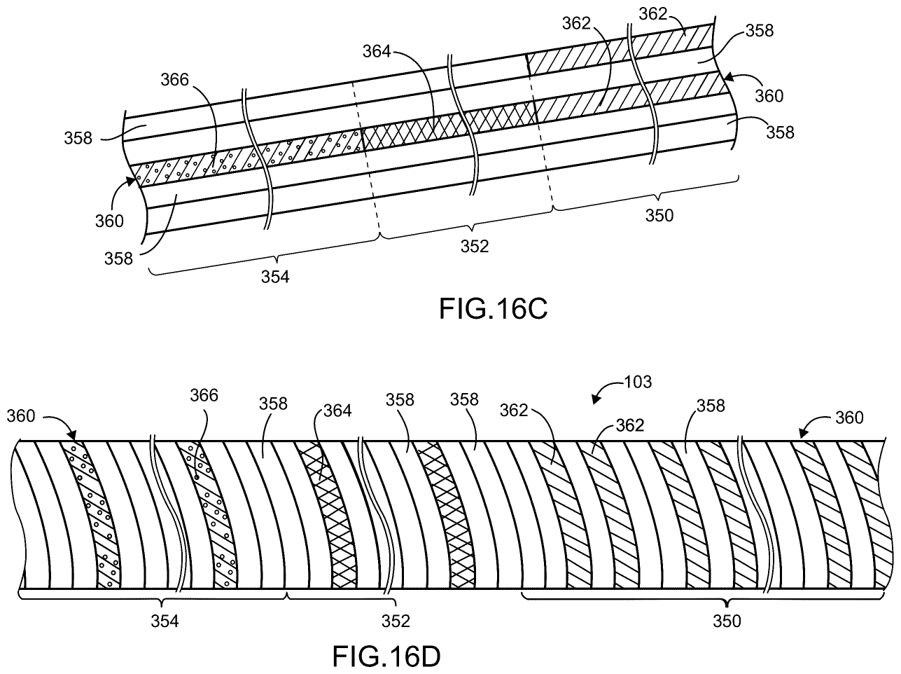

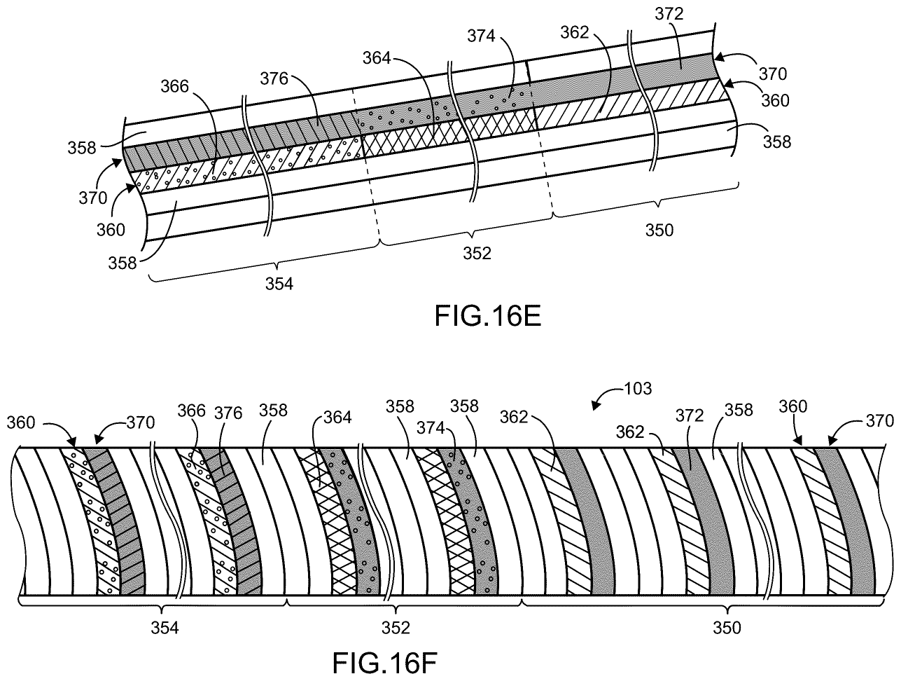

FIGS. 16A and 16B illustrate an additional example of a construction of a tube 103 for use with the devices described herein. FIG. 16A is a sectional view of a number of strands 358, 362, 364, 366 that are joined to form a tubular section, as depicted in FIG. 16B. In FIG. 16B, the tubular section 103 includes a plurality of material sections that extend in a continuous spiral over the tube section 103, where one material section 360 that extends from region 350 through regions 352 and 354 changes materials at each region. In one example, the material section 360 comprises a reinforcement material section as it continuously and spirally extends over multiple regions. In addition, the structural properties of each region can be selectively engineered based the individual materials 362, 364, and 366. For example, to increase flexibility from a proximal to distal direction of a device, the first region 350 can comprise a material 362 having a hardness/durometer that is greater than material 364 in adjacent/second region 352.

In additional variations, the third region 354 can comprise a material 366 having a durometer/hardness that is less than a hardness/durometer material 364. It is noted that material sections adjacent to material section 360 (e.g., 358) can include any number of materials as discussed herein. However, in some variations of the devices described herein, material section 360 comprises a hardness/durometer that is greater than a hardness/durometer of each adjacent material section 358 in the respective region. For example, in first region 350, material section 360 can comprise a material 362 that has a durometer/hardness greater than the material of each adjacent materials section 358 in that same region (i.e., region 350).

Similarly, in additional variations, this construction can be repeated in regions 352 and 354, where material 364 comprises a greater durometer than the materials in the adjacent material sections within that region, and material 366 comprises a greater durometer than the materials in the adjacent material sections within that region. In such an example, material section 360 can effectively function as a continuous torque coil within the tube member 130 (at least across any two sections) but can have a hardness/durometer that changes or is gradually decreased as required by the application. In those situations that require a catheter to reach distal regions, the catheter can be constructed via the tubular member 103 to have increasing flexibility towards a distal region while still employing a continuous torque coil that also decreases in flexibility.

In yet an additional variation, the constructions shown in FIGS. 16A and 16B can comprise configurations where material section 360 comprises a durometer/hardness that is lower than the adjacent material sections 358 and/or decreases in each region (350, 352, 354).

FIGS. 16C and 16D illustrate another example of a construction of a tube 103 for use with the devices described herein. FIG. 16C shows a sectional view of a number of strands 358, 362, 364, 366 that are joined to form a tubular section, as depicted in FIG. 16D. However, the area that forms region 350 includes two sections of material 362 having the same durometer/hardness. This construction is shown in FIG. 16D, where the tubular section 103 includes a plurality of material sections that extend in a continuous spiral over the tube section 103, where one material section 360 that extends from region 350 through regions 352 and 354 changes materials at each region, while region 350 includes two spirally wound materials 362 having the same durometer/hardness. In such a configuration, first region 350, material section 360 comprises a material 362 that has a durometer/hardness different than the material of each adjacent materials section 358 in that same region (i.e., region 350) but equal to another material section material 362. As noted above, the durometer/hardness of material 362 can be greater or less than the adjacent sections.

FIGS. 16E and 16F show another potential example of a construction of a tube 103 for use with the devices described herein. FIG. 16E illustrates a sectional view of a number of strands 358, 362, 364, 366 that are joined to form a tubular section as depicted in FIGS. 16A and 16B above, with an additional material section 370 that contains materials 372, 374, and 376 in respective sections 350, 352, and 354. FIG. 16E shows the tubular section 103 formed from the configuration of FIG. 16E, where the plurality of material sections that extend in a continuous spiral over the tube section 103 and one material section 360 serves as a reinforcement material section, where the hardness/durometer of materials 362, 364, 366 in that material section extends across regions 350, through regions 352 and 354 and changes materials at each region. However, the construction shown in FIGS. 16E and 16F also show a material section 370 having materials 372, 374, and 376 that are lower than or equal to the hardness/durometers of adjacent materials in material section 358. In additional variations, the hardness/durometers of materials 372, 374, and 376 can decrease respectively, in sections 350, 352, and 354. While material section 360 is shown to be directly spirally adjacent to material section 370, additional variations include spacing of the highest and lowest durometer materials as opposed to being directly adjacent.

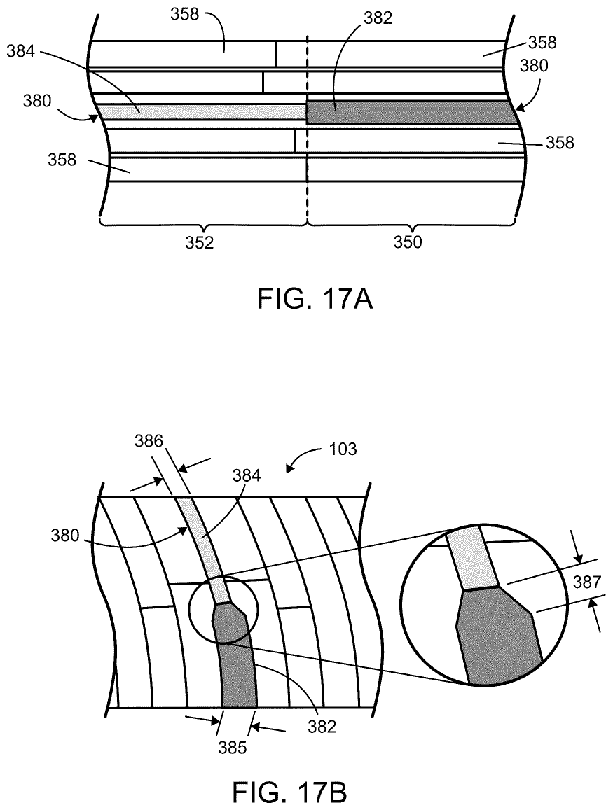

FIGS. 17A and 17B illustrate another example of customizing material sections to adjust the structural characteristics of a tube member 103 and/or device as described above. FIG. 17A illustrates a series of material sections 358 adjacent to a transition material section 380. As noted above, material section 358 can comprise any variety of materials that are desired for adjusting the characteristics of the device. FIGS. 17A and 17B illustrate a transition material section 380, which includes a first width 385 corresponding to a width of the transition region 380 in the first region 350. The transition material section includes a second width 386, corresponding to a width of the transition region 380 in the second region. As discussed herein, not only can the widths vary, but the height/depth of the material can be changed. Moreover, FIG. 17B illustrates that material section 380 initially comprises a first material 382 and then transitions to a second material 384. However, in additional variations, the material section can comprise a single material that changes from a first width 385 to a second width 386. Additional variations of the design can include a transition region that changes from a smaller dimension to a larger dimension in a distal direction along the tubing.

As discussed above, FIG. 17A illustrates the state of the tubular portion prior to the material sections being joined in a spiral configuration. Once joined into a tubular structure 103, as shown in FIG. 17B, the materials on either side of the thinner region of the transition material section 380 fill in to form a completely sealed joint between adjacent material sections. As shown, the first region 350 can include a section where a width of the transition material section is consistent, and at the start of the second region 352, the transition material section 380 steps down at a step-down region 387. Variations of this configuration include a length of the step-down region 387 being less than the width of the larger transition material section. In another variation of this configuration, the transition material section 380 can include a first material 382 in the first region 350 and a second material 384 in the second region 352. As described above, the transition material section 380 can comprise materials to permit the section 380 to function as a torque coil, e.g., where the hardness/durometer of the transition section is greater than adjacent material sections. Alternatively, the transition material section can comprise a hardness/durometer that is less than adjacent material sections. In even further variations, a single tubular structure 103 can include multiple step-down regions for different material sections. Alternatively, or in combination, section 380, or any material that has a greater hardness/durometer than an adjacent material, functions as a type of “push coil” where this increased hardness/durometer material is helically formed in the catheter tubing and reinforces the tubing when pushed from a proximal location.

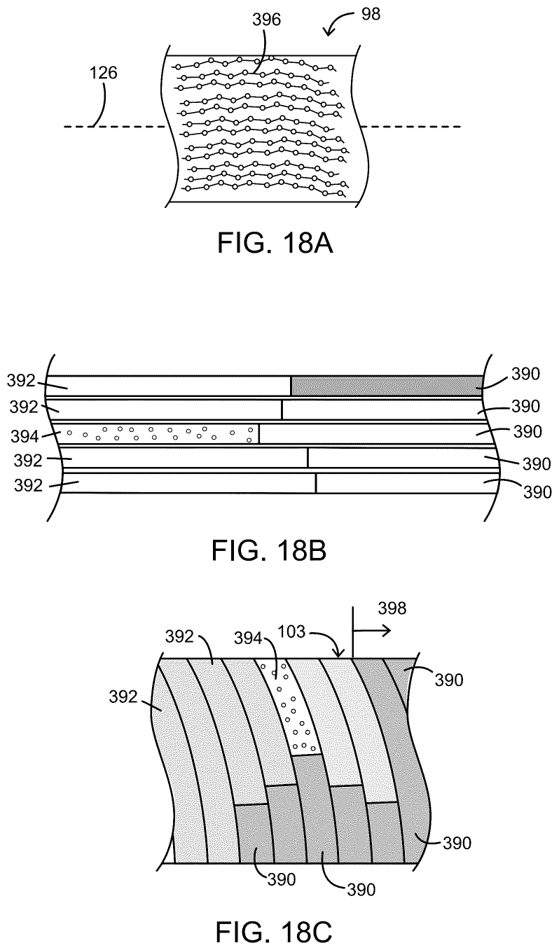

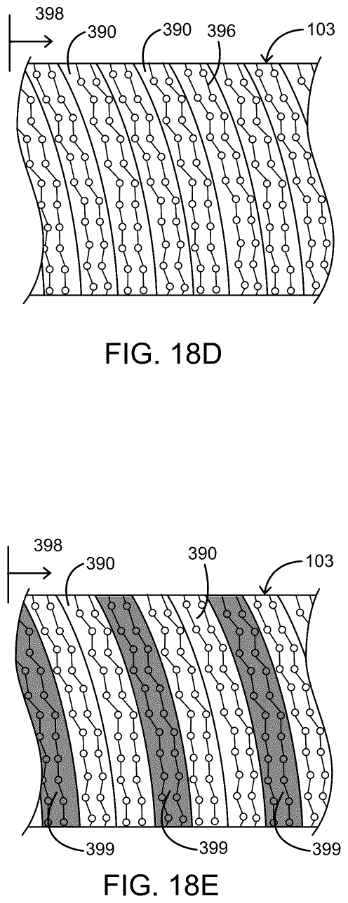

FIGS. 18B to 18F illustrate another design variation for use in composite tubing sections. In a traditional catheter construction, as shown in FIG. 18A, the use of an extruded material 98 aligns the polymer chains 396 along an axial length of the tube in alignment with an axis 126 of the material 98. Therefore, flexure of a conventional stiff tube causes bending in a normal direction against a backbone of the polymer chain 396. Excessive bending can lead to fracture of the polymer 98. One benefit of constructing tube 103 from one or more polymers that have a high clastic modulus/stiffness is that the polymer chains 396 are oriented in a spiral direction about the tube structure 103. For example, in the variations as shown in FIGS. 18C to and 18E, the polymer chains 396 extend in a helical pattern, which allows increased flexure of the tube structure 103 and reduces the risk of fracture of the material due to bending of the tube structure 103. FIG. 18B illustrates one example of a plurality of material sections where material 390 is joined to materials 392 and 394. These materials are sealingly joined to form tube structure 103 of FIG. 18C, where the material sections extend spirally along an axial length of the tube structure 103 to form a wall that can optionally be incorporated into one of the devices described herein. In this variation, the material sections in a first region (designated in the direction shown by arrow 398) are formed from a first polymer 390 such that a polymer chain extends spirally in that region following the spiral of the material section as it is wrapped to form the tube 103, as shown in FIG. 18D. Having a construction as shown in FIG. 18C, where in the first region, the plurality of material sections comprises a first polymer 390 that has a high modulus/stiffness adjacent to a second region where each of the polymers 392, 394 comprise a lower modulus/stiffness than material 390 allows a device design with a proximal region that is stiff but more resistant to fracture. FIG. 18E shows another variation of a construction 103 where the first region 398 formed from one or more material sections extending spirally and comprising a single polymer 390 with a similar polymer 399 having the same structural properties as the first polymer 390 but is distinguishable/identifiable from the first polymer 390 (e.g., via color, surface texture, markers, radiographic, etc.). In such a construction, the material properties of section 398 are the same, but the tubular structure 103 can be uniquely identified by the distinguishable pattern either visually and/or mechanically) resulting from materials 390 and 399 so that a caregiver can identify the tubing relative to other tubing. For example, when used in a catheter positioned in a patient via a femoral or radial artery, the caregiver is able to distinguish the catheter having the features shown in FIG. 18E relative to other catheters when region 398 extends from the patient's body. It is understood that section 398 will comprise at least two distinguishable material sections, 390, 399 having similar or the same structural properties, while the remaining region of the catheter (e.g., as shown in FIG. 18C) could have additional material sections as discussed herein. Alternatively, the remainder of the catheter could also have a conventional extruded construction.

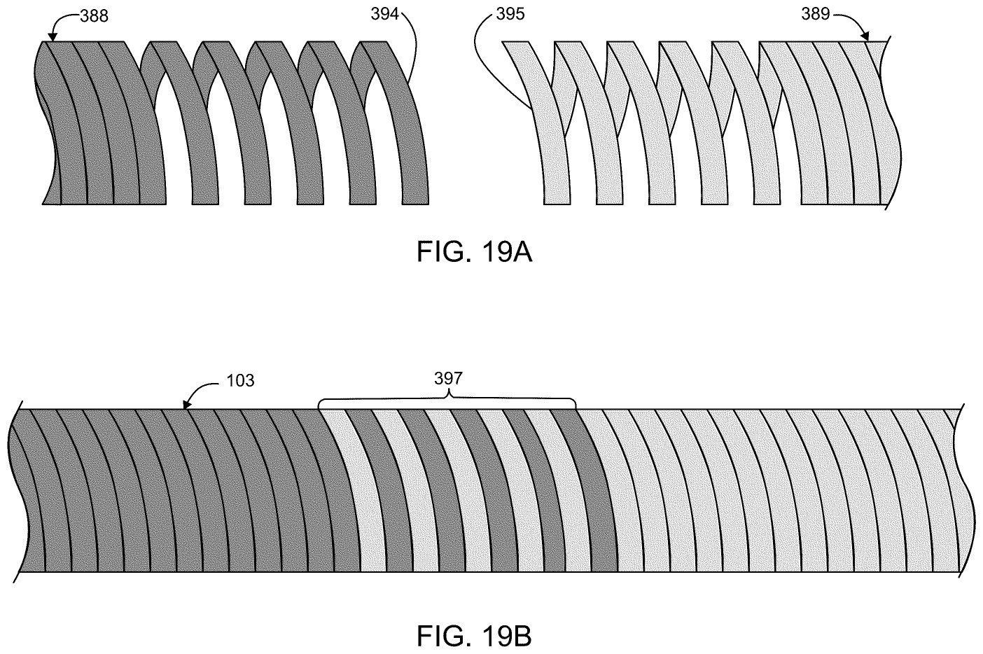

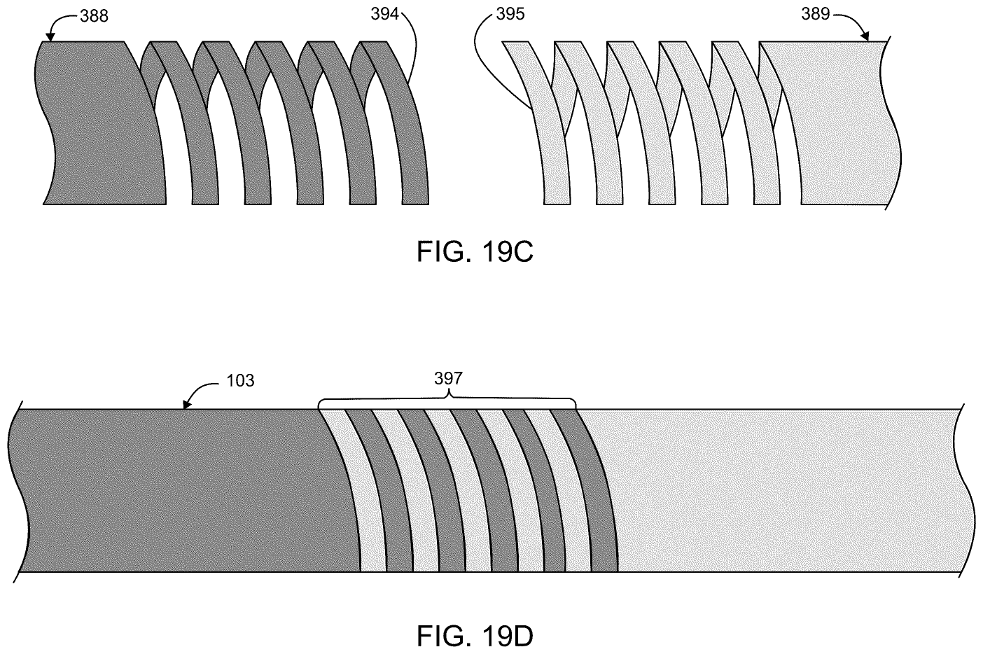

FIGS. 19A to 19D illustrate another variation of a composite tube 103 for use in the devices described herein. In this variation, FIG. 19A shows a first material, tube 388, that can be conventionally extruded as a common single-lumen tubing. As shown, the tube 388 is separately spaced from a second conventionally formed material, tube 389. Each material tube 388 389 can have separate properties, as discussed above. In addition, the tubes 388 389 can be respectively cut (e.g., via laser cutting) to create a spiral or other helical pattern 394, 395. FIG. 19B illustrates the tubes 388 and 389 of FIG. 19A sealingly joined together such that the different properties of each material section provide a transition section 397 similar to those discussed above. FIG. 19C illustrates a variation where a first material tube 388 and second material tube 389 are continuous and then cut to form a helical patterns 394, 395 that are ultimately joined to form transition section 397, as shown in FIG. 19D.

The ability to incorporate softer materials with a relatively harder material, as shown in the above figures, allows for improved custom selection of device properties. Moreover, the ability to transition continuously spiral material sections into different materials as well as controllably stepping down in width allows for a drastic improvement in catheter design. The ability to change materials as described herein provides manufacturers with the ability to change a greater number of catheter design elements to fine-tune catheter construction to a degree that was previously unavailable with conventional catheter construction.

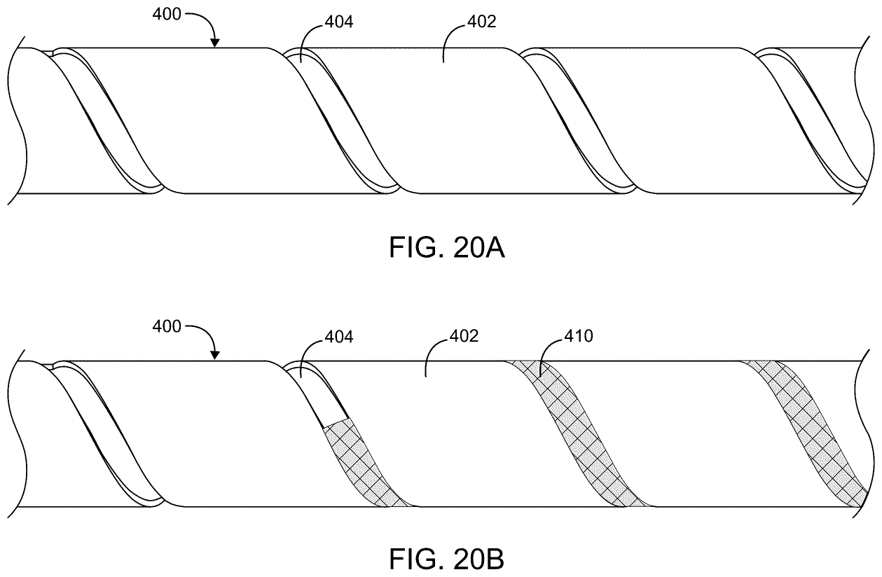

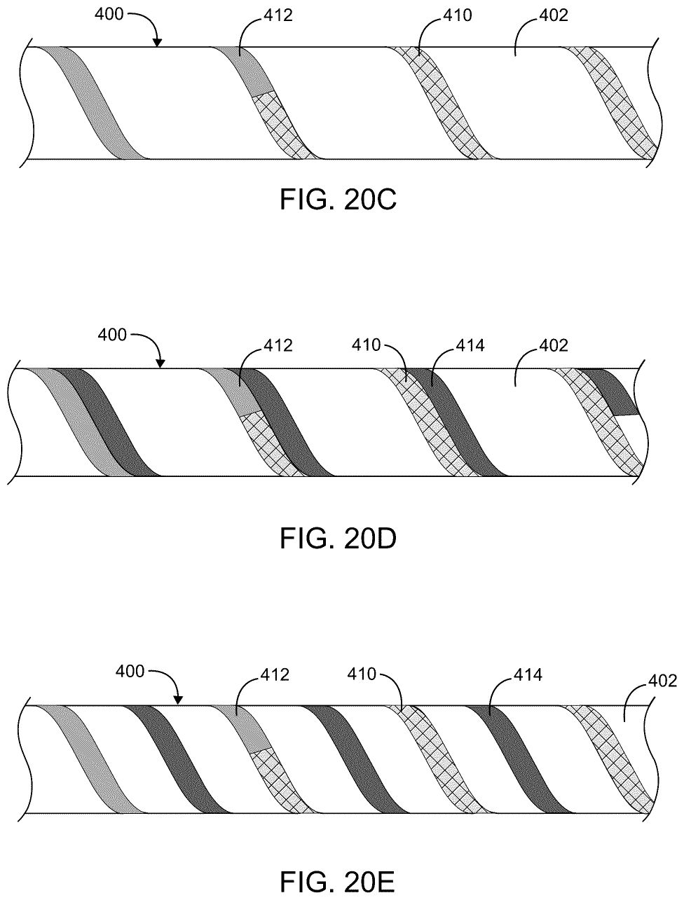

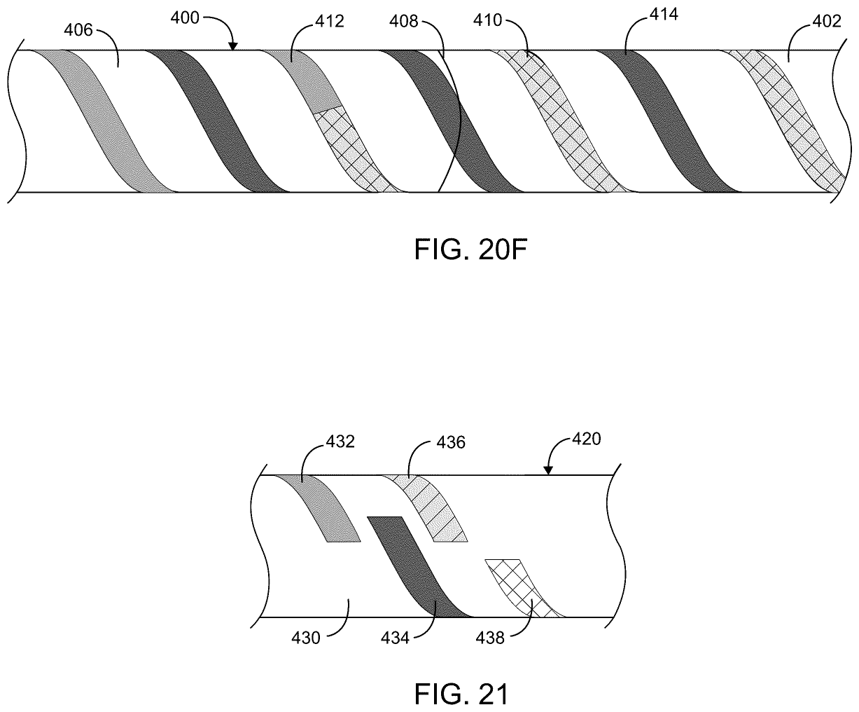

FIG. 20A to 20F illustrates another variation of fabrication of a composite tube. As shown in FIG. 20A, a tube 400 can be formed from a base material 400 (e.g., by extrusion, 3D printing, or any other fabrication process). The tube 400 can be altered or formed to have spiral grooves (that extend through the wall) or slots (i.e., cuts that do not extend through the entirety of the wall). Next, as shown in FIG. 20B, a material such as a polymer or other material, is joined to the tube material 402 within the groove 404 to form a material section 410 that extends within the tube 400. FIG. 20C shows a second material forming a second material, section 412, that is positioned within the groove and joined to an end of the first material, section 410. Accordingly, FIG. 20C shows a tube structure 400 having three different materials 402, 410, and 412.

FIG. 20D shows a variation similar to that shown above, but where material sections 410 and 412 are adjacent to a different material section 414. FIG. 20E illustrates two joined material sections, 410 and 412, with a material section that is spaced apart, 414. FIG. 20F shows another variation where the tube structure 400 can be formed from a plurality of tubes having different materials 402, 406 with different structural properties or different distinguishable properties. In such a case, a tube comprising material 402 can be joined to a tube comprising material 406 at a juncture 408, and the composite tube 400 can be altered as discussed above in FIG. 20A.

FIG. 21 illustrates another variation of a composite tube 420 that is formed with non-overlapping material sections 432, 434, 436, 438 such that the material or polymer of the tube 430 separates the material sections. As shown, the material sections can be axially spaced (e.g., 434 is axially spaced from 432 and 436). Furthermore, the ends of the material sections can overlap (e.g., 434, 432, 436) or the ends can be spaced as well (e.g., 438). The construction 420 shown in FIG. 24 can be fabricated by entirely by the use of material sections where material 430 is wound with the remaining material sections. Alternatively, the composite tube 420 can comprise a polymeric (formed from an extrusion) or other tube comprising material 430 and mechanically altered with slots or grooves to allow for insertion of the material sections 432, 434, 436, 438.

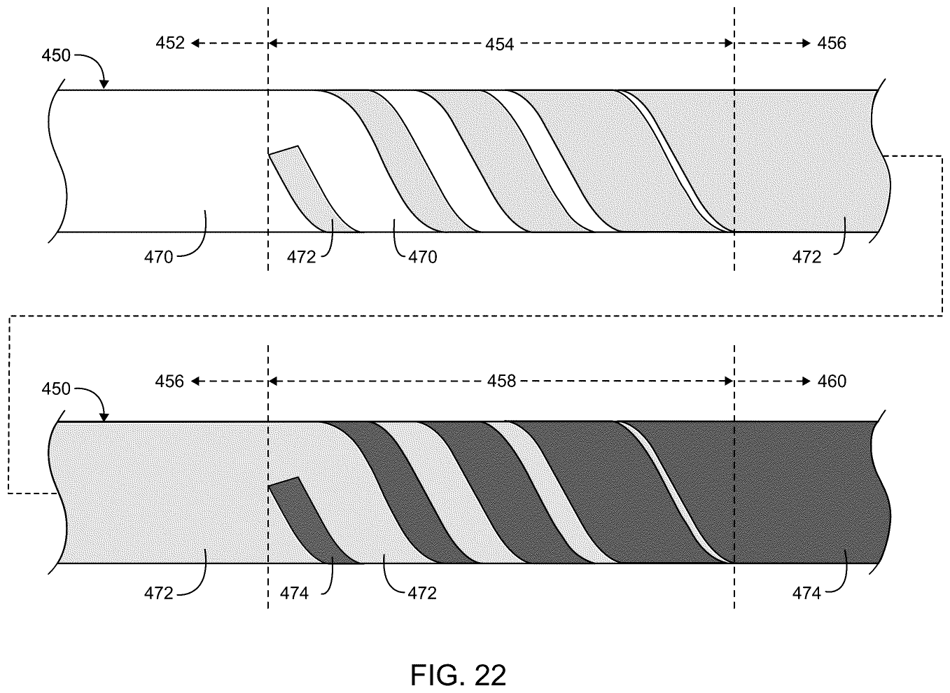

FIG. 22 illustrates another variation of a composite tubing 450 that can be formed using any process described herein or used in manufacturing of tubing. FIG. 22 illustrates the tubing 450 having a first region that comprises a single material 470, where the first region 452 is adjacent to a second region 454, where a material section 472 comprising a second material begins in a spiral pattern. In the second region, 452, the first material forms a first material, section 470, that decreases in a width (as measured axially) while the second material, section 472, increases in width until a third region, 456 that, is fully formed from the second material 472. The composite tubing continues to a fourth region, 458, where a third material, section 472, causes formation of material section 472, which decreases in width as the third material, section 474, increases in width. As discussed herein, each material 470, 472, 474 will have different properties (e.g., different structural properties and/or visually distinguishable properties), allowing for the overall property of the composite tube 450 to be customized. FIG. 22 also shows that the third material 474 continues in a fifth region, 460, that is entirely formed from the third material 474. It is noted that the widths, spacing, and spiral wind of the material sections shown in FIG. 22 are for illustrative purposes only and can be combined with any variation discussed herein. In addition, in regions 452, 456, and/or 460 in, the composite tube 450 can be formed from an extruded tubes which are mechanically altered in the second region 454 and fourth region 458. Alternatively, regions 452, 456, and/or 460 can be spirally wound.

It is noted that the polymer strands disclosed herein can extend in a helical manner about the inner braid/coil or support structure. In additional variation, the polymer strands can be aligned in a lengthwise manner with an axis of the catheter and wrapped about the support structure to form a catheter section. Any number of manufacturing practices can be used to produce the catheter constructions of the present disclosure. For example, the strands can be directly wrapped on a liner/braid construction and then fused together to form a catheter construction; 2) the strands can be wrapped over a tube and fused, then transferred to the remaining components to produce a catheter construction; and/or 3) the strands can be produced as a flat construction (either fused together, extruded, molded, or otherwise formed) and then ribbon assembly wrapped and fused onto a liner/braid. The devices described herein can also be constructed using a 3-d printing process.

It is understood that any manufacturing process is within the scope of this disclosure and should not be limiting upon any claimed structure to any claims relating to composite polymer tubes or catheter constructions.

As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

1. A catheter tubing comprising:
a tubular structure extending along an axial length and having a passageway;
a tubular layer forming at least a portion of a wall of the tubular structure and comprising a plurality of material sections extending spirally along at least a portion of the axial length, where each material section is joined to an adjacent material section;
wherein the plurality of material sections include at least a first material section and a second material section, the first material section comprises a first structural property and the second material section comprises a second structural property, where the first structural property differs from the second structural property;
at least one lumen extending within at least one of the plurality of material sections, where the at least one lumen permits fluid flow therethrough.

2. The catheter tubing of claim 1, further comprising at least one opening in the tubular layer, where the at least one opening is fluidly coupled to the at least one lumen.

3. The catheter tubing of claim 2, further comprising a balloon having a balloon material over the at least one opening, where the balloon material is affixed to the tubular layer such that pressurization of the at least one lumen causes expansion of the balloon.

4. The catheter tubing of claim 2, further comprising a balloon material extending along the tubular layer having an interior surface at least partially joined to an exterior of the tubular layer, wherein such that when pressurized by the at least one opening in the tubular layer, the balloon material expands away from the exterior of the tubular layer to assume a balloon shape on the tubular structure.

5. The catheter tubing of claim 1, wherein the plurality of material sections comprises a plurality of tubular extensions.

6. (canceled)

7. The catheter tubing of claim 1, wherein the plurality of material sections comprises a first material section comprising an expandable material and where the at least one lumen comprises a first lumen within the first material section, wherein pressurization of the first lumen causes the first material section to expand radially outward from the tubular layer to forms a spiral structure extending along at least a portion of the axial length of the tubular layer.

8.-9. (canceled)

10. A medical tubing comprising:
an elongate body extending along an axial length; and
a plurality of material sections extending spirally along at least a first portion of the axial length to form at least a portion of a wall of the elongate body, the plurality of material sections including a first material section;
wherein the first material section includes a first functional lumen extending therein, such that pressurization of the first functional lumen causes the first material section to expand radially outward from the plurality of material sections to form an expanded spiral shape on the elongate body.

11. The medical tubing of claim 11, wherein the plurality of material sections comprises at least a second material section extending spirally over at least a second portion of the axial length.

12. The medical tubing of claim 11, where the second material section includes a second functional lumen in fluid communication with at least one opening in a surface of the elongate body.

13. The medical tubing of claim 12, further comprising a balloon material covering the at least one opening.

14. The medical tubing of claim 10, wherein the expanded spiral shape comprises a plurality of turns having a spacing.

15. The medical tubing of claim 14, wherein the spacing of the plurality of turns prevents fluid flow between the plurality of turns, such that the expanded spiral shape comprises an occlusion region when expanded.

16. The medical tubing of claim 14, wherein the spacing of the plurality of turns permits permit fluid flow between the plurality of turns.

17. (canceled)

18. A catheter tubing comprising:
a tubular structure extending along an axial length and having a passageway;
a composite layer forming at least a portion of a wall of the tubular structure and comprising a plurality of tubing extensions extending spirally along at least a portion of the axial length, where at least one of the plurality of tubing extensions includes a lumen extending therein.

19. The catheter tubing of claim 18, wherein at least a first tubing extension from the plurality of tubing extensions changes structural properties over a length of the first tubing extension.

20. The catheter tubing of claim 18, wherein a structural property of at least two of the plurality of tubing extensions is different.

21. The catheter tubing of claim 18, wherein the plurality of tubing extensions includes a solid tubular extension.

22. The catheter tubing of claim 18, wherein the plurality of tubing extensions includes a tubing extension having one or more openings in an exterior surface of the composite layer.

23. The catheter tubing of claim 22, further comprising a balloon material exterior to the one or more openings, such that pressurization through the one or more openings causes the balloon material to expand radially outward from the tubular structure.

24. The catheter tubing of claim 18, wherein the plurality of tubing extensions includes an expandable tubing extension with a functional lumen such that pressurization of the functional lumen causes the expandable tubing extension to expand radially away from the composite layer to form an expanded spiral shape about an exterior of the tubular structure.

25.-26. (canceled)

27. A catheter tubing comprising:
a tubular structure having a wall and a passageway extending in the tubular structure; and
a material section extending spirally in a portion of the wall over a first axial length, where the material section comprises a functional lumen extending therein along the first axial length, where the functional lumen is fluidly isolated from the passageway such that the functional lumen can be pressurized independently from the passageway.

28. The catheter tubing of claim 28, wherein the material section comprises a first structural characteristic that is different from an axially adjacent material in the portion of the wall.

29. The catheter tubing of claim 27, where the functional lumen terminates in at least one opening at a surface of the tubular structure.

30. The catheter tubing of claim 29, further comprising an expandable material at an exterior of the tubular structure and fluidly coupled to the at least one opening, such that pressurization of the functional lumen causes the expandable material to radially expand away from a surface of the tubular structure.

31. The catheter tubing of claim 27, wherein the material section is configured to expand outwardly from the tubular structure upon pressurization of the functional lumen such that the material section forms an expanded spiral shape over an exterior of the tubular structure.

32. The catheter tubing of claim 27, wherein a material property of the material section changes over a portion of the first axial length.

33. The catheter tubing of claim 27, wherein the wall of the tubular structure further includes a plurality of additional material sections extending spirally in the portion of the wall.
